(19) United States
(12) Patent Application Publication
(10) Pub. No.: US 2020/0217837 A1
(43) Pub. Date: Jul. 9, 2020

(54) CHROMATIN PROTECTIVE THERAPEUTICS AND CHROMATIN HETEROGENEITY

(71) Applicant: Northwestern University, Evanston, IL (US)

(72) Inventors: Vadim Backman, Chicago, IL (US); Igal Szleifer, Evanston, IL (US); Luay Almassalha, Evanston, IL (US); John Chandler, Evanston, IL (US); Greta Bauer, Evanston, IL (US); Scott Gladstein, Evanston, IL (US)

(21) Appl. No.: 16/318,037

(22) PCT Filed: Jul. 14, 2017
(86) PCT No.: PCT/US2017/042168

§ 371 Date: Jan. 15, 2019

Related U.S. Application Data
(60) Provisional application No. 62/362,940, filed on Jul. 15, 2016.

(51) Int. Cl. G01N 33/50; G01N 33/68; G01N 33/574

(52) U.S. Cl. CPC G01N 33/5017; A61K 45/06; G01N 33/574; G01N 33/6875

(57) ABSTRACT

Provided herein are chromatin protection therapeutics (CPTs) and methods of targeting chromatin heterogeneity for the treatment of cancer therewith. In particular compositions and methods are provided that target physical variations in chromatin topology, reduce chromatic heterogeneity, and treat cancer or inhibit the development of resistance to other cancer therapeutics.

### CROSS-REFERENCE TO RELATED APPLICATION

The present application claims priority to U.S. Provisional Patent Application Ser. No. 62/362,940 filed Jul. 15, 2016, which is hereby incorporated by reference in its entirety.

### STATEMENT REGARDING FEDERAL FUNDING

This invention was made with government support under CBET1249311 awarded by the National Science Foundation. The government has certain rights in the invention.

### FIELD

Provided herein are chromatin protection therapeutics (CPTs) and methods of targeting chromatin heterogeneity for the treatment of cancer therewith. In particular compositions and methods are provided that target physical variations in chromatin topology, reduce chromatic heterogeneity, and treat cancer or inhibit the development of resistance to other cancer therapeutics.

### BACKGROUND

Research has historically shown a broad plasticity in the origin of tumors and their functions, with significant heterogeneity observed in both morphologies and functional capabilities. Largely unknown, however, are the mechanisms by which these variations occur and how these events influence tumor formation and behavior. Contemporary views on the origin of tumors focus mainly on the role of particular sets of driver transformations, mutational or epigenetic, with the occurrence of the observed heterogeneity as an accidental byproduct of oncogenesis.

### SUMMARY

Provided herein are chromatin protection therapeutics (CPTs) and methods of targeting chromatin heterogeneity for the treatment of cancer therewith. In particular compositions and methods are provided that target physical variations in chromatin topology, reduce chromatic heterogeneity, and treat cancer or inhibit the development of resistance to other cancer therapeutics.

In some embodiments, provided herein are compositions comprising a chromatin protective therapeutic (CPT), wherein the CPT reduces chromatin heterogeneity and/or inhibits increases in chromatin heterogeneity. In some embodiments, the CPT is formulated as a pharmaceutical composition. In some embodiments, CPTs are co-formulated with a cancer chemotherapeutic.

In some embodiments, provided herein are methods of preventing cancer and/or tumor formation in a subject comprising administering to the subject a chromatin protective therapeutic (CPT), wherein the CPT reduces chromatin heterogeneity and/or inhibits increases in chromatin heterogeneity, thereby reducing the likelihood of cancer and/or tumor formation in the subject.

In some embodiments, provided herein are methods of preventing chemotherapeutic resistance and/or immune evasion in a subject being treated for cancer comprising administering to the subject a chromatin protective therapeutic (CPT), wherein the CPT reduces chromatin heterogeneity and/or inhibits increases in chromatin heterogeneity, thereby reducing the likelihood of cancer and/or tumor formation in the subject.

In some embodiments, a CPT is co-administered with a chemotherapeutic. In some embodiments, the CPT and the chemotherapeutic are administered simultaneously. In some embodiments, the CPT and the chemotherapeutic are co-formulated. In some embodiments, the CPT and the chemotherapeutic are administered sequentially. In some embodiments, the CPT is administered (e.g., initial administration, final administration, etc.) at least 1 hour (e.g., 2 hours, 4 hours, 8 hours, 12 hours, 24 hours, 2 days, 3, days, 4 days, 1 week, 2 weeks, 4 weeks, or more, or ranges therebetween) prior to the chemotherapeutic. In some embodiments, the chemotherapeutic is administered (e.g., initial administration, final administration, etc.) at least 1 hour (e.g., 2 hours, 4 hours, 8 hours, 12 hours, 24 hours, 2 days, 3, days, 4 days, 1 week, 2 weeks, 4 weeks, or more, or ranges therebetween) prior to the CPT.

In some embodiments, provided herein are methods of monitoring over time the treatment and/or prevention of cancer, the likelihood of a subject developing cancer, and/or the progression of cancer in a subject, comprising: (a) measuring chromatin heterogeneity in a population of cells from the subject at a first time point; (b) measuring the chromatin heterogeneity in a similar population of cells from the subject at a second time point; (c) comparing the chromatin heterogeneity at the first and second time points, wherein: (i) a decrease in chromatin heterogeneity indicates cancer is being treated or prevented successfully, the likelihood of the subject developing cancer is reduced, and/or cancer is not progressing in the subject; or (ii) an increase in chromatin heterogeneity indicates cancer not being treated or prevented successfully, the likelihood of the subject developing cancer is increased, and/or cancer is progressing in the subject. In some embodiments, methods further comprise administering a chromatin protective therapeutic between steps (a) and (b). In some embodiments, the population of cells is obtained by biopsy.

In some embodiments, provided herein are methods of identifying a chromatin protective therapeutic (CPT), comprising: (a) measuring chromatin heterogeneity in a population of cells at a first time point; (b) administering a test agent to the population of cells; (c) measuring the chromatin heterogeneity in the population of cells at a second time point; (d) comparing the chromatin heterogeneity at the first and second time points, wherein a decrease in chromatin heterogeneity indicates the test agent is active as a CPT. In some embodiments, chromatic heterogeneity is measured by live-cell partial wave spectroscopic microscopy.

In some embodiments, provided herein are methods of screening a library of test agents to identify a chromatin protective therapeutic (CPT), comprising: (a) measuring chromatin heterogeneity of multiple populations of cells at a first time point; (b) administering different test agents to each of the populations of cells; (c) measuring the chromatin heterogeneity in each of the population of cells at a second time point; (d) comparing the chromatin heterogeneity at the first and second time points, wherein a decrease in chromatin heterogeneity indicates a test agent is active as a CPT. In some embodiments, chromatic heterogeneity is measured by high-throughput live-cell partial wave spectroscopic microscopy.

### BRIEF DESCRIPTION OF THE DRAWINGS

FIGS. 1A-B. Tumor formation models. (FIG. 1A) Clonal expansion secondary to perturbation is classically defined as the cause of tumorigenesis. Clonal expansion often well characterizes hematopoietic tumors and pediatric tumors, but often fails to explain the underlying heterogeneity observed in solid organ tumors. In the cancer stem cell model, tumors arise due to the formation of stem cells that give rise to new tumor with multiple subtypes, allowing for a partial heterogeneity in cell origin within a tumor. Greater Genomic Landscape focuses on the general feature of multicellular systems to change their function in the face of stress. In GGL, tumors arise due to the probability of a population arriving at a cancer state due to the selection of a large distribution of cell subpopulations (and functions) and from increased information sampling that it produces. (FIG. 1B) Five predominant subpopulations within a tissue for a given cell type are considered. Following from a gamma distribution, small changes in the heterogeneity (scale parameter) result in large deviations in the number of subpopulations. While the average population and tissue function does not change significantly, the total number of possible states (and functions) has increased.

FIG. 2. Available information space for cellular subpopulations. (A) Conservative estimates in the heterogeneity of subpopulations for different types of cellular variance, assuming subpopulations are distinct but largely share the same features. If cells each express 1000 proteins and only 10 are different between subpopulations (99% overlap in function), then 10ⁿ potential variations are possible. Likewise, if each subpopulation has 5 distinct mutations, ˜3×10¹⁷ genetic states are possible for 25 distinct subpopulations. Often overlooked, however, is the effect of varying the physical configurations of chromatin. Even a 1% difference in the organizational topology would allow 3.3×10⁵⁷ potential responses for 25 subpopulations. If physical heterogeneity increases to 4%, this increases to 3.7×10⁷², an ˜10¹⁵ increase in possible responses.

FIGS. 3A-C. Physical Structure of Chromatin and Gene Regulation. (FIG. 3A) Microarray analysis of gene expression for differentially expressed genes shows a correlation in the induction of expression (R²=0.63) with concomitant suppression of genes (R²=0.75) and underlying heterogeneity. (FIG. 3B) Differential Transcriptional activity (DTA=% Up−% Down) shows the accompanying increase in total expression for genes as a function of the physical nanoarchitecture. Comparisons were made between the initial state and all other groups. R² for each comparison >0.78, and 0.70 overall. (FIG. 3C) Network Heterogeneity increases with increase in the heterogeneity of the chromatin nanoarchitecture. Cluster domains for 22 GO processes that contain at least 10 genes with their respective functions are shown. Each point represents an ontological process, with the intensity defined by the standard deviation of relative expression for genes within that process for CV SE conditions compared to the condition above CV EGF vs. A-KD SE. A higher ΔL_{d} state is associated with an increased heterogeneity of gene expression within a network, in particular for transcriptional regulation, multicellular development, signal transduction, and cellular proliferation.

FIGS. 4A-H. Chemoevasion, chromatin protection therapies, and chromatin heterogeneity. (FIG. 4A) LC-PWS microscopy quantification of chromatin heterogeneity for A2780 and A2780.m248 cells treated with Paclitaxel. Cells treated with traditional chemotherapeutics (e.g., pactaxol, 5-FU, oxilaplatin, etc.) all display increase in heterogeneity after treatment. This effect is independent of cell line and chemotherapeutic mechanism. (FIG. 4B) Representative field of view of untreated A2780 control cells imaged by LC-PWS microscopy and (FIG. 4C) representative field of view for A2780 cells treated with 5 nM paclitaxel for 24 hours showing an increase in chromatin heterogeneity after treatment. (FIG. 4D) LC-PWS microscopy quantification of chromatin heterogeneity for A2780 and A2780.m248 cells treated with Celecoxib, a selective COX-2 non-steroidal anti-inflammatory drug. Neo-adjuvants such as celecoxib (FIGS. 4E,F) and digoxin, demonstrate a homogenization of chromatin within minutes of cell treatment. (FIG. 4G, panel i)) Transmission image of untreated control A2780 and (FIG. 4g, panel ii)) transmission imaging of untreated control A2780.m248 cells. (FIG. 4G, panels iii) and iv)) For both cell populations, cells treated with combination digoxin (150 nm) and paclitaxel (5 nm) demonstrate a ˜90% reduction in cell mass. (FIG. 4H) Efficacy of chromatin protective therapies varies from cell to cell, but drugs that demonstrate a homogenization of chromatin organization for a particular cell type demonstrate an increase in chemotherapeutic efficacy. Likewise, all chemotherapeutic compounds in all cells measured increased heterogeneity of chromatin over time.

FIG. 5. Orthographic z-axis projection of molecular dynamics simulation of chromatin as a 10 nm “beads on a string” polymer capturing (Panel A) differentially compacted and (Panel B) diffusely compacted chromatin. Scale bar represents 100 nm. Calculated transmission microscope image captured by (Panel C) conventional bright-field microscope from differentially compacted chromatin in (Panel A) and (Panel D) of diffusely compacted chromatin in (Panel B). Images were produced by calculating the average mass density at each pixel and a Gaussian PSF of 250 nm was applied to simulate a conventional microscope. (Panels E&F) Calculation of Σ captured by live cell PWS from differentially compacted chromatin in (Panel A) and diffusely compacted chromatin in (Panel B). Σ was calculated directly from the distribution of mass within configurations shown in A&B with Σ=0.01-0.065. (Panel G) Representative pseudo-colored Live cell PWS image of HeLa cells with 63× oil immersion lens, NA=1.4 with Σ scaled to range between 0.0125 to 0.065. (Panel H) Co-localization of fluorescence with Live cell PWS image showing mitochondria, nuclei), and mitochondria-nucleus overlap. Scale bar is 20 μm. (Panels J&K) Representative pseudo-colored Live cell PWS image of (Panel J) HeLa cells and (Panel K) MES-SA cells demonstrating the capacity to capture nanoscopic information from dozens of nuclei in seconds with Σ scaled to range between 0.01 to 0.05 in J and 0.01 to 0.065 in K.

FIG. 6. Hoechst excitation induces rapid transformation of chromatin nano-architecture. (Panel A) Pseudo-colored Live cell PWS image of Hoechst 33342-stained HeLa cells before and after excitation of the dye with UV light. Transformation of chromatin occurs across the whole nucleus within seconds and no repair is observed even after 15 minutes (Panel B) Hoechst stained and mock-stained cells before excitation and (Panel C) the same mock stained and Hoechst stained cells after UV irradiation. (Panel D) Minimal (mock) and significant (Hoechst) γH2A.x antibody accumulation. (Panel E) Distribution of chromatin transformation after UV excitation for Hoechst and Mock stained cells. (Panels F&G) TEM images of Control and Hoechst stained cells confirming nanoscale fragmentation of the chromatin nano-architecture in fixed cells. All pseudo-colored images scaled between Σ=0.01-0.065. Scale bars are all 15 μm.

FIG. 7. Live cell PWS uniquely detects nano-architectural transformation resulting from Hoechst incubation and excitation. Live cell PWS (Panel A) and Phase Contrast Panel (B) cells pre-incubation, 15-minute post-incubation, Hoechst fluorescent image, and after excitation. (Panel C) Change in the autocorrelation function of Live cell PWS intensity. Hoechst transforms chromatin into a more globally heterogeneous structure. Live cell PWS images are scaled between Σ=0.01-0.065. Scale bars are all 15 μm.

FIGS. 8A-D. Live cell PWS detects dynamics of nano-architectural transformation under normal and UV-irradiated conditions. (FIG. 8A) Representative field of view displaying 7 HeLa cells imaged in ˜15 seconds using a 63× oil immersion lens, NA=1.4 with Σ scaled to range between 0.01 to 0.065 over 30 minutes of imaging. (FIG. 8B) Representative field of view displaying 7 HeLa cells exposed continuously to UV-light imaged in ˜22 seconds using a 63× oil immersion lens, NA=1.4 with Σ scaled to range between 0.01 to 0.065 over 30 minutes of imaging. (FIG. 8C) Inset from field of view in (FIG. 8A) showing the time evolution of two nuclei. Interestingly, chromatin organization is rapidly evolving in time, showing that even at steady state, the underlying structure changes. (FIG. 8D) Inset from field of view in (FIG. 8B) showing the time evolution of one nuclei under UV-illumination. Under UV exposure, homogeneous micron-scale domains form within chromatin, lacking their original higher-order structure.

FIG. 9. Live cell PWS detects dynamics of nano-architectural transformation under normal and UV-irradiated conditions. (Panel A) Kymograph (with the x-axis representing a linear cross-section in x-y plane and the y-axis showing changes over time) representing the temporal evolution of chromatin of a cell exposed to continuous UV-light. Nanoscopically homogenous, micron-scale domains form within the nucleus after ˜5 min of exposure with an overall arrest in structural dynamics. (Panel B) Kymograph representing the temporal evolution of chromatin of a cell under normal conditions. Under normal conditions, the nanoscale topology of chromatin is highly dynamic, with continuous transitions in structure occurring throughout the nucleus. (Panel C) Quantitative analysis of nanoscale structure of chromatin of cells under normal conditions and exposed to UV-light for 30 minutes. Exposure to UV-light induces overall homogenization of chromatin nano-architecture within minutes. Error bars represent standard error. Scale Bar is 5 μm.

FIGS. 10A-D. Mitochondrial membrane potential (ΔΨm) is a direct, rapid regulator of chromatin compaction. FIG. 10A) Flow Cytometry showing a 10-fold decrease in Hela cell TMRE fluorescence after 10 μM CCCP treatment (p<0.015) and no significant change in CHO cell fluorescence. Row FIG. 10B) HeLa and FIG. 10C) CHO cells before and 15 minutes after CCCP treatment. FIG. 10D) Quantification of the nuclear nano-architecture change in HeLa and CHO cells before and after treatment (HeLa=31 cells, 6 replicates and CHO=159 cells, 5 replicates) with standard error bars. Depletion of ΔΨm induces decompaction and homogenization of HeLa but not CHO chromatin. Live cell PWS images are scaled between Σ=0.01-0.065. Scale bars are all 15 μm, arrows indicate nuclei.

FIGS. 11A-B. (FIG. 11A) Effect of CPTs on mean nuclear Σ on A2780.m248 ovarian cancer cells. (FIG. 11B) Percent cell death of A2780.m248 ovarian cancer cells in th presence of various combinations of chemotherapeutic and CPTs.

FIG. 12. PWS image of live HeLa cells. Pseudo-color: heterogeneity of macro-molecular density with sensitivity to length scales from 20 to 200 nm. N—nuclei.

FIGS. 13A-C. Predictive modeling of transcriptional heterogeneity due to chromatin heterogeneity. (FIG. 13A) MSA model of gene transcription rate (Ref. 1c; incorporated by reference in its entirety), ε, is a non-monotonic function of molecular crowding within the interaction volume, where transcriptional molecular reactions take place, due to the competition of two effects of crowding: increased molecular binding rates—this facilitates transcription through the stabilization of transcription complexes—and decreased diffusion, which lowers the probability of formation of the complexes. bottom curve: an active gene; top curve: a suppressed gene; both are normalized so that ε(φ=0)=1. (FIG. 13B) Gene expression (E) sensitivity to an increase in chromatin heterogeneity (Se=(dE/E)/(dD/D)) was assayed by an mRNA profiling array (2445 genes) in HT29 colon cancer cell lines. Circles: microarray data. Each data point is an average of 100 genes with similar initial expression (E). Black line: MSA predictions (i.e. BD+MC+analytical model, Eq. for Se is below) based on the parameters derived from the simulations in Ref. 1c. The model depends on: D (measured by PWS), M_{f}—the genomic length of the fractal chromatin globule (Ref. 3c; incorporated by reference in its entirety), Lᵢₙ—the size of the interaction volume relative to a base pair (Ref. 1c), L—gene length in bp. (FIG. 13C) Higher chromatin heterogeneity (i.e. D↑,Σ↑) leads to transcriptional divergence (c, left) (The interquartile range of E in the excess of the initial range.) and inter-cellular gene expression heterogeneity (c, right) (The average inter-colony standard deviation of a change in gene expression in response to transcription.

FIGS. 14A-D. Variations in chromatin folding modulate transcriptional heterogeneity. (FIG. 14A) Structural alterations due to taxol treatment (Paclitaxel or Docetaxel) in contrast to digoxin for five cell line models (A2780, M248, MDA-MB-231, MES-SA, MX2). Chemotherapeutic intervention increases while CPT agent (digoxin) decreases chromatin folding. Error bars: S.E. of 5 different cell lines. (FIG. 14B) Intercellular and (FIG. 14C) intra-network transcriptional heterogeneity increases in cells treated with chemotherapy and decreases in cells treated with CPTs for critical biological processes: (1) cell cycle, (2) apoptosis, (3) proliferation, (4) transcription, (5) signaling, (6) differentiation, (7) glycolysis, (8) translation, (9) ion transport, (10) metabolism, (11) oxidation/reduction, (12) stress response, and (13) nucleosome assembly. Circle size: the number of genes within a network/process. Color: % change in transcription heterogeneity compared to controls. (FIG. 14D) Representative live cell PWS images: digoxin reduces chromatin heterogeneity. Arrow: nuclei.

FIGS. 15A-I. (FIG. 15A) PWS images of a 24-hour time course of HCT116 colon cancer cell lines after treatment with chemotherapy drug (Oxaliplatin), CPT (Celecoxib), and their combination. The addition of a CPT shows 98% cancer cell death within 48 hours. Similar results were obtained with other CPTs, chemotherapy drugs, and cell lines (lung, ovarian, breast, leiomyosarcoma, and liver cancers). (FIG. 15B) Chromatin heterogeneity Σ is increased in cancer cells that survive chemo-therapy (IC₅₀, 48 hour time point). (FIG. 15C) Σ↓, within 30 min in cells treated with CPT agents valproic acid (VPA), digoxin, and celecoxib (p<0.01) but not in cells treated with non-CPT sulindac. (FIG. 15D) CPT significantly increases the efficacy of chemotherapeutic agents independent of the molecular pathway of the chemotherapy drug. Mild CPTs (5-10% D↓) are less effective than moderate CPTs (10%-20% D↓). The addition of a CPT can achieve 100% cancer cell death. Key: Docetaxel (D), Docetaxel+Digoxin (DD), Docetaxel+Celecoxib (DC), Paclitaxel (P), Paclitaxel+VPA (PV), Paclitaxel+Celecoxib (PC), Paclitaxel+Digoxin (PD), Oxaliplatin (O), Oxaliplatin+Aspirin (OA), Oxaliplatin+Celecoxib. (FIG. 15E) CPTs alone do not induce apoptosis (A2780 cells, 48 hour timepoint). (FIG. 15F) Cell death added by CPT+chemotherapy co-treatment (OC) compared to chemotherapy alone is proportional to the efficacy of CPTs to reduce D (measured by PWS). CPT Index=reduction in D x reduction in intercellular variability in D. (FIG. 15G) Co-treatment of A2780 cells with paclitaxel (Pac) and CPT celecoxib (Pac+Cel) results in ˜100% cancer cell death even for the 0.01% of the IC₅₀ dose. (FIG. 15H) The reduction of chromatin heterogeneity by CPT (celecoxib) is greater for cells with the more abnormal chromatin structure (high initial Σ˜D) (r²=0.96). (FIG. 15I) Validation of CPT agent (9-ING-41) in vivo on the pancreatic ductal carcinoma PDX model. Left: 9-ING-41 decreases Σ in multiple cancer cell lines. Right: Animals were treated i.p. 3× a week with a chemotherapy drug gemcitabine (10 mg/kg) and/or 9-ING-41 (40 mg/kg). The CPT+gemcitabine co-treatment produced shrinkage in tumor volume <4% of the initial size.

FIG. 16. Comparison of molecular and physico-chemical regulators of the chromatin nanoarchitecture. Molecular regulators on chromatin folding: SWI/SNF inhibition (sh-RNA BRG-1 Kd), histone methyltransferase inhibition (UNC0638, UNC1999, GSK-126), HDAC inhibition (sh-RNA Sirt6 Kd, VPA), DNA methyltransferase inhibition (SGI-110), and cohesin inhibition (sh-RNA SA-1 Kd). Physiochemical modulation: potassium depletion (Digoxin) and glycogen synthase kinase 3b inhibition (9-ING-41). Physico-chemical modulation is more potent in comparison to known chromatin modulators.

FIG. 17. Chromatin modulation by CPT agents does not depend on pathway-specific chromatin remodeling. Comparison of the effects of a CPT agent (Celecoxib) on WT colon cancer HCT-116 cells in comparison to Sirt6 kd and Brg1 kd shows no difference in higher order chromatin folding between these pathways. This indicates that for CPTs such as celecoxib the observed global chromatin de-heterogenization is independent of known chromatin modifying pathways. All measurements were performed on >5 replicates per condition.

FIGS. 18A-B. Tables depicting the effect of various CPT compounds on chromatin heterogeneity in various cancer cell lines.

FIG. 19. Table depicting whether various CPT compounds synergize with chemotherapeutics, to enhance cell death and/or allow for reduced chemotherapeutic dose, in various cancer cell lines.

FIG. 20. Table depicting compounds tested that had no CPT effect.

### DEFINITIONS

The terminology used herein is for the purpose of describing the particular embodiments only, and is not intended to limit the scope of the embodiments described herein. Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a CPT” is a reference to one or more CPTs to immunotherapy and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition (e.g., cancer).

As used herein, the terms “pharmaceutical agent” and “therapeutic agent” refer to a compound, peptide, macromolecule, or other entity that is administered to a subject to elicit a desired biological response. A pharmaceutical agent may be a “drug” or another entity which is biologically active in a human being or other mammal, locally and/or systemically. Examples of drugs are disclosed in the Merck Index and the Physicians Desk Reference, the entire disclosures of which are incorporated by reference herein for all purposes.

As used herein, the term “pharmaceutical formulation” refers to at least one pharmaceutical agent and/or microbial agent in combination with one or more additional components that assist in rendering the agent(s) suitable for achieving the desired effect upon administration to a subject. The pharmaceutical formulation may include one or more additives, for example pharmaceutically acceptable excipients, carriers, penetration enhancers, coatings, stabilizers, buffers or other materials physically associated with the pharmaceutical/microbial agent to enhance the administration, release (e.g., timing of release), deliverability, bioavailability, effectiveness, etc. of the dosage form. The formulation may be, for example, a liquid, a suspension, a solid, a nanoparticle, emulsion, micelle, ointment, gel, emulsion, coating, etc. A pharmaceutical formulation may contain a single agent or multiple agents (e.g., a CPT and chemotherapeutic or immunotherapeutic).

As used herein, the term “co-administration” refers to the administration of at least two agents (e.g., a CPT and a cancer therapeutic) or therapies to a subject. In some embodiments, the co-administration of two or more agents/therapies is concurrent. In other embodiments, the co-administration of two or more agents/therapies is sequential (e.g., a first agent/therapy is administered prior to a second agent/therapy).

The terms “effective dose” and “therapeutic dose” refer to an amount of an agent (e.g., an chemotherapeutic, an immunotherapeutic, a CPT, etc.), that results in the reduction of symptoms in a patient or results in a desired biological outcome. In certain embodiments, an effective dose or therapeutic dose is sufficient to treat or reduce symptoms of a disease or condition.

As used herein, the term “subtherapeutic dose” refers to an amount or dose of a therapeutic agent (e.g., chemotherapeutic, immunotherapeiutic, etc.) that is lower than the conventional dose administered to a subject alone (e.g., for the same indication, by the same administration route). In particular, it refers to an amount or dose of a therapeutic agent which has no effect or only a slight effect when used alone. In particular, the subtherapeutic dose may be 90%, 80%, 70%, 60%, 50%, 40%, 30%, 20% or 10% of the conventional dose.

As used herein, an “immune response” refers to the action of a cell of the immune system (e.g., T lymphocytes, B lymphocytes, natural killer (NK) cells, macrophages, eosinophils, mast cells, dendritic cells, neutrophils, etc.) and soluble macromolecules produced by any of these cells or the liver (including Abs, cytokines, and complement) that results in selective targeting, binding to, damage to, destruction of, and/or elimination from a subject of invading pathogens, cells or tissues infected with pathogens, or cancerous or other abnormal cells.

As used herein, the term “immunotherapy” refers to the treatment or prevention of a disease or condition (e.g., cancer) by a method comprising inducing, enhancing, suppressing or otherwise modifying an immune response.

As used herein, “potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “antibody” refers to a whole antibody molecule or a fragment thereof (e.g., fragments such as Fab, Fab′, and F(ab′)2), it may be a polyclonal or monoclonal antibody, a chimeric antibody, a humanized antibody, a human antibody, etc.

### DETAILED DESCRIPTION

Provided herein are chromatin protection therapeutics (CPTs) and methods of targeting chromatin heterogeneity for the treatment of cancer therewith. In particular compositions and methods are provided that target physical variations in chromatin topology, reduce chromatic heterogeneity, and treat cancer or inhibit the development of resistance to other cancer therapeutics.

Experiments conducted during development of embodiments herein indicate that tumors form due to heterogeneous adaptive selection in response to environmental stress through intrinsic genomic sampling of their available genomic/epigenomic information (i.e., the Greater Genomic Landscape). This heterogeneous adaptive sampling is pharmacologically-targetable using therapeutics (e.g., small molecules, peptide, antibodies, etc.) that limit the broad access to the Greater Genomic Landscape. Provided herein are chromatin protective therapeutics (CPTs), which target physical variations in chromatin topology, and methods of designing, developing, screening, and testing such CPTs. By targeting the physical organization of chromatin, CPTs reduce the overall information space available for sampling, and limit the formation of tumors, the development of drug-resistant phenotypes, etc. Thereby, CPTs provide tumor prevention/treatment and/or act as adjuncts to other therapies (e.g., for therapy-resistant cancer).

Evolution has traditionally been studied as the set of mechanisms that confer heritable traits from parents to their progeny. In this view, evolutionary sampling confers traits that can be advantageous to the progeny under the appropriate circumstances. As such, under stress conditions that favor a given set of traits, the populations with those traits will clonally expand and predominate. In multicellular organisms, the distinction between progeny and evolutionary fitness becomes blurred. Intuitively, clonal selection of cell populations within a tissue can be advantageous to the whole organism, but are not reproductively heritable to the multicellular progeny. For the cell population at the tissue level, the discovered adaptions are not classically ‘selective’ but ‘capacitive,’ e.g., the resulting heterogeneous population confers an advantage to a plurality of traits since a broader distribution can help in the face of new stresses. However, by definition, this increase in traits fundamentally changes the tissue over time.

The most studied model of this evolution-driven functional transformation in humans is cancer (refs. 1-4; incorporated by reference in their entireties). Largely unknown, however, are the mechanisms by which adaptive sampling occurs and how these events could result in the formation of tumors. Results have historically shown a plasticity in the origin of tumors, with heterogeneous mutational and epigenetic events occurring throughout a challenged organ preceding an eventual pathological expansion (refs. 2, 5, 6; incorporated by reference in their entireties). Furthermore, tissues under constant energetic and replicative pressures account for the demonstrable majority of tumors (ref. 7a; incorporated by reference in its entirety). These observations, however, do not fully explain the broad distribution of molecular events that can precipitate tumor formation. Contemporary views on the origin of tumors derive from the monoclonal expansion of cells (e.g., tumor stem cells, clonal selection due to mutations or chromosome instability) into a lesion before the occurrence of the observed heterogeneous acceleration (ref 1a; incorporated by reference in its entirety). This view, however, does not explain the functional diversity in tissues under non-perturbed conditions even within cells of the same lineage (ref. 8a; incorporated by reference in its entirety).

Experiments conducted during development of embodiments herein indicate that tumors form due to heterogeneous adaptive selection in response to environmental stress through intrinsic genomic sampling mechanisms; although the embodiments herein are not limited to any undelying mechanism and an understanding of the mechanism of action is not necessary to practice such embodiments. In some embodiments, eukaryotic cells intrinsically explore their available genomic information, in response to stress under normal conditions, in real time, and this occurs long before the formation of a cancerous lesion. This information, the Greater Genomic Landscape (GGL), is the available distribution of functional states: the current functions of the cell (e.g., proteomic, metabolic) and possible future states (e.g., genes that can be expressed/repressed or mutated). In essence, the GGL merges critical traits of information theory and evolutionary biology to explain tumorigenesis as something other than an accidental byproduct, but a consequence of multicellular fitness. Specifically, the intrinsically encoded exploration of genomic information is a main adaptive advantage of multi-cellularity and occurs primarily at three levels and time scales: (1) post-translational proteomic (rapid−seconds/hours), (2) epigenomic (intermediate−minutes/days), and (3) mutational (days-weeks-years). For example, in epigenomic sampling the normal chromatin nanoenvironment helps restrict cells to a relatively small niche within the genomic information space formed by the estimated ˜20,000 human protein-coding genes; however, deviations in chromatin structure, such as those observed in cancer cells, facilitate a greater genomic exploration. The GGL does not refer to the well-established concept of cancer genome landscapes, which refers to the set of genes altered in carcinogenesis. Rather, the GGL refers to the ability of a cell to sample its genome.

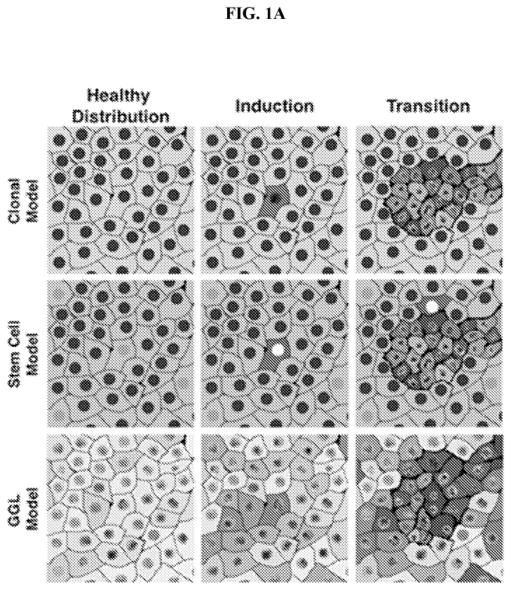

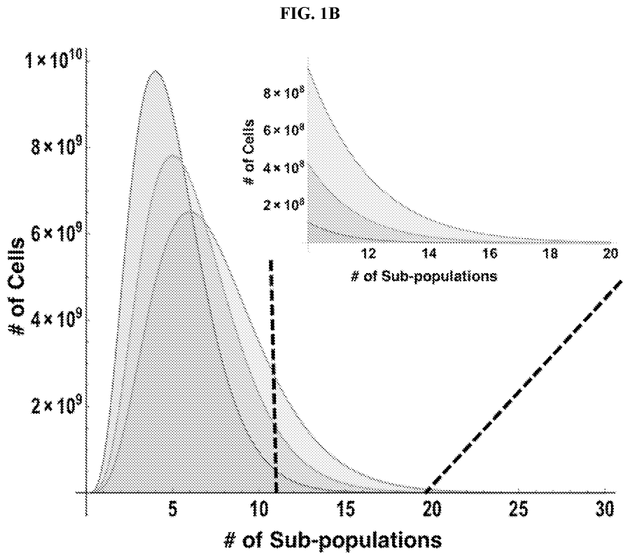

Cells comprise intrinsically encoded mechanisms of information sampling for the three levels of genomic information. At the proteomic level, there are numerous non-transcriptional ways to alter cellular function. For instance, studies of yeast under stress demonstrate that eukaryotic cells employ a plurality of strategies to respond to conditions, including varying abundance and location of proteins (and mRNA), leading to a heterogeneity of initial conditions and variability of response to stress (refs. 10, 11; incorporated by reference in their entireties). At the epigenomic level, there are both enzymatic and non-enzymatic ways to alter the information space. In tumorigenesis, there are numerous demonstrations of chromatin remodeling enzymes being critical drivers in chemoevasion and tumor formation. However, there is also an often overlooked level of epigenetic heterogeneity, which is to vary the initial configurations of chromatin structure to change accessibility and probability of expression for genes from cell to cell. Critically, both the proteomic and epigenetic mechanisms happen at time scales that are faster than the division of cells, allowing cells to discover new adaptions during exposure to stress. The presence of rare subpopulations occurs at significant levels even while maintaining an “average” population (FIG. 1). An increase in the heterogeneity of subpopulations does not necessarily transform the overall tissue function, but it may have a profound effect on the information space available to respond to stress conditions. Classically, this is considered at the time scale of cell division, with mutational alterations as the predominant mode of increasing the genomic information space by creating inherently new potential functions. In this way, mutational transformation is also the classical example of tumor heterogeneity, but occurs at time scales that are challenging to target pharmacologically.

Consequently, repeated and multidimensional stressors will select for cells with traits that enhance the capacity to search the GGL, not just for a particular set of proteomic pathways or initial traits which in single cell systems. As a result, each perturbation increases the heterogeneity of the underlying tissues by favoring a broader distribution of semi-unique states and cells that have the greatest plasticity (e.g., capacity to search for new functions). Over time, this differential sampling of the genome produces an increasingly diverse population, commonly observed as the detection of overt tumors as they by definition have unique features. It is this tissue heterogeneity and intrinsic plasticity that acts as a conserved evolutionary mechanism that favors more exploratory cells in eukaryotic systems, resulting in tumor formation through the increased probability of proliferative configurations.

Tumor formation is an evolution-driven information-sampling problem arising as stress induces the population of cells to sample the information coded within their genomes and proteomes to collectively maintain tissue function. The origins of these stresses are innumerable (e.g., alcohol, smoking, infections, etc.) and as such, the tissue does not a priori know what mechanism of evasion will be successful. Instead, cells carry a limited repertoire of encoded proteins that include intrinsic samplers to rapidly and probabilistically search the GGL for solutions to maintain the underlying function of the tissue. This occurs not by just rapidly inducing all genes, but by combinatorically exploring the information space encoded across numerous subpopulations. Within an individual cell, these intrinsic samplers initiate a probabilistic search response at both the proteomic (e.g., post-translational modification) and genomic (e.g., chromatin remodeling, mutational transformation) levels. The cells that fail during this sampling under stress undergo apoptosis or mitotic arrest (e.g., after a few hours).

There is a distribution of time scales during which the levels of stress response occur. In particular, sampling is relatively rapid in comparison to mechanisms of cellular clearance, i.e. apoptosis and immune-clearance. Evidence of this separation of timescales has been observed previously, even indicating possible transition states between death and survival (ref 12a; incorporated by reference in its entirety). Irreversible commitment to apoptosis occurs over the course of several hours, while proteomic transformation and chromatin remodeling are very rapid (<a few minutes). This indicates that irreversible commitment to apoptosis is delayed in order to give cells time to find stress evasion mechanisms. Without this complementary intrinsic sampling mechanism, tissues would fail under mild perturbation from unique stressors.

Central convergence points exist between exploration, apoptosis, and cellular arrest. As such, one regulator of intrinsic sampling of the GGL is mitochondrial membrane potential, Ψₘ. Mitochondria are ubiquitously implicated in diseases, specifically diseases of aging, e.g., tumors, neurodegeneration, and atherosclerosis (ref. 13a; incorporated by reference in its entirety). Beyond this central association, disruption of Ψₘ has been shown to regulate the epigenetic structure of chromatin, molecular signaling cascades, and post-translational modification of cytoplasmic proteins (ref. 13a; incorporated by reference in its entirety). Furthermore, processes directly linked to Ψₘ include apoptosis, proliferation, and senescence (ref 14a; incorporated by reference in its entirety). Consequently, in some embodiments, Ψₘ provides a central barometer of cellular fitness, mediating sampling, apoptosis, and senescence concurrently. In some embodiments, disruption of Ψₘ simultaneously induces proteomic and genomic exploration, initiates the apoptosome, and potentiates cell cycle arrest (refs. 15, 16; incorporated by reference in its entirety). If the stressor is not resolved, either extrinsically or intrinsically, cells commit to apoptosis to limit their use of resources, saving resources for the remaining cells.

It is contemplated that the evolutionary selection of more robust samplers and an increasingly heterogeneous population of cells occurs primarily for two reasons; although the embodiments herein are not limited to any particular mechanism of action and an understanding of the mechanism of action is not necessary to practice such embodiments. First, continuous maintenance of many traits is energetically unfavorable for an individual cell. Secondly, more robust samplers and a greater number of initial states increases the likelihood of finding traits that prevent tissue failure during duress. With each perturbation event, selective pressures transform tissues by increasingly favoring a broader distribution of cellular configurations and cells with increased plasticity. Over time, this accelerates the evasive fitness and increases the cellular heterogeneity present within the affected tissue (ref. 1a; incorporated by reference in its entirety).

With Ψₘ as a barometer of fitness, evolutionary selection produces cells with the following combinations of features; cells that (1) more rapidly and thoroughly explore the genomic space, (2) have previously acquired a higher stress tolerance, (3) preferentially arrest to extend survival, and (4) have a broad distribution of initial states (FIG. 1).

Differential exploration selects for numerous populations of cells within a healthy (or unhealthy) tissue under the same stress. For example, at least two different mechanisms favor cell survival in the presence of a toxin: (1) inactivating genes involved in the apoptotic cascade or (2) creating proteins that expel the stressor. As a result, repeated or multidimensional perturbations do not select for one trait, but instead broaden the distribution of initial cell states and favor more elastic samplers. This feature is conserved in normal tissues, and is not an adaption unique to carcinogenesis.

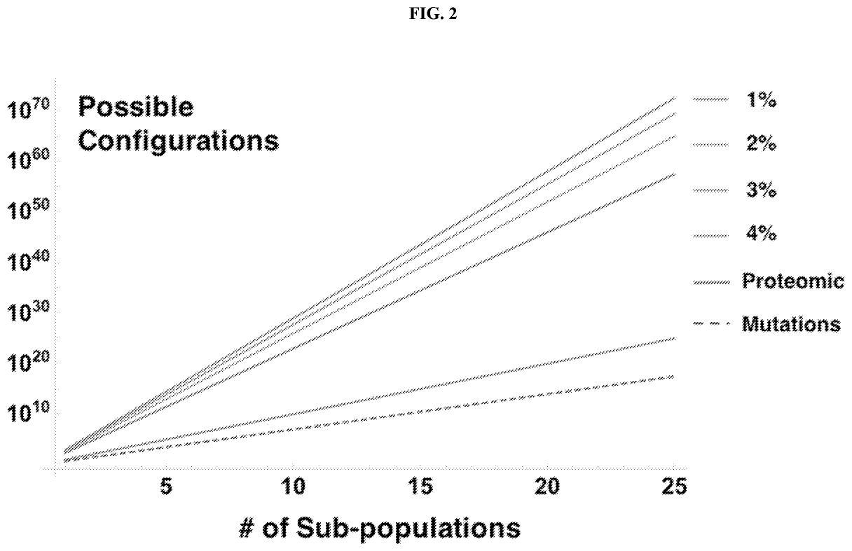

Evolutionary sampling of the GGL is a critical feature of tumorigenesis and normal tissue function; therefore, one mechanism to increase the exploration of the GGL and enhance the chance of cellular survival during stress conditions is to delay the irreversible commitment to apoptosis, thereby extending the duration of exploration and allowing the search of more possible evasive combinations (ref. 18a; incorporated by reference in its entirety). A second mechanism is the transformation of chromatin remodeling enzymes to increase the efficiency of combinatorial searches in response to stress (refs. 19-20; incorporated by reference in their entireties). A third mechanism is to broaden the heterogeneity of chromatin structure of the cellular population (e.g., vary the configurations to increase coverage across the entire population (ref. 21a; incorporated by reference in its entirety). By increasing the distribution of chromatin organization across cells, each cell within the population has a different initial configuration state that produces a semi-unique exploration, enhancing the total information space (FIG. 2). As a result, five subpopulations would have ˜3×10¹¹ unique genomic configurations with only 1% variation in chromatin topology compared to 10⁵ proteomic states with a similar level of proteomic variability.

This indicates that the underlying heterogeneity of chromatin organization (and the ability to modulate the structure) has a disproportionate influence on tissue function, cellular diversity, and fitness. Even without taking into consideration additional influences such as cell communication, distinct cellular populations, and the time evolution of chromatin structure, this indicates an overwhelming influence of physical organization of chromatin on the probability of tumor formation. While not every potential configuration would be attempted in every stress, it is the distribution (e.g., the total number of possibilities) that assist the tissue over long periods of time, as it allows tissues to function across many different exposures. The tradeoff is that increased variation increases the probability of acquiring negative traits. Interestingly, the observation of physical heterogeneity of chromatin (e.g., variations in fractal dimension) as a prognostic marker in cancer is well conserved in solid tumors and may be a proxy for the underlying information space within a tissue (e.g., higher fractal dimension produces greater variability in structure) (ref. 22a; incorporated by reference in its entirety).

Cancer is not a disease of a few specific mutations but involves the dysregulation, both mutational and transcriptional, of the complex interactions of hundreds of genes. Currently no existing platform allows for predictable transcriptional modulation of this many genes simultaneously. Although studies of chromatin structure have identified numerous molecular regulators of nucleosomal compaction and the role of genome compartmentalization that may help explain the transcription patterns of individual genes, largely absent from the field has been an understanding of the role of the highly dense and complex physical nanoenvironment within chromatin on transcriptional molecular reactions. Since transcriptional interactions are chemical reactions, they depend on the local physical nanoenvironment, which in turn, depends on the physical pattern of chromatin folding at supra-nucleosomal length scales.

Experiments were conducted during development of embodiments herein to develop tools to modulate the chromatin nanoenvironment for whole-scale transcriptional engineering for cancer prevention and treatment. In particular, transcriptional diversity is shown to play a major role in carcinogenesis by allowing cancer cells to survive and continue developing new hallmarks despite unfavorable internal (e.g. hypoxia, immune system) or external (e.g. chemotherapy, immunotherapy) interactions. Chemotherapy provides a particularly significant example. Despite advances in chemo- and immunotherapy, for many solid cancers complete remission is still rare (Ref. 2c; incorporated by reference in its entirety). Although immuno- and targeted therapies are able to improve survival for a few specific cancer sub-types, in the majority of cases the added progression-free survival is counted in months. Even if a tumor undergoes remission, the rate of relapse is high with the recurrent tumors frequently developing multi-drug resistance (Ref. 4c; incorporated by reference in its entirety). A key cause behind the emergence of resistance is tumor heterogeneity and tumor cells' ability to change their gene expression patterns and adapt (Refs. 5c-6c; incorporated by reference in their entireties). New gene mutations are not necessary for drug resistance, and a change in the expression of existing genes due to transcriptional diversity may suffice (Refs. 7c-8c; incorporated by reference in their entireties). Consequently, heterogeneity of gene expression within a tumor is a critical factor in primary drug resistance, as well as the emergence of new drug-resistant clones (acquired resistance) (Ref 9c; incorporated by reference in its entirety).

Experiments conducted during development of embodiments herein demonstrate that abnormal chromatin nanoenvironment plays a critical role in facilitating cancer cells' ability to dynamically change their global gene expression patterns, explore a greater genomic landscape and consequently adapt to and develop resistance to chemotherapy (Refs 10c-12c; incorporated by reference in their entireties). A class of cancer therapeutics has been developed, based on the physico-chemical modulation of chromatin nanoenvironment, termed chromatin protection therapy (CPT). CPTs reduce cancer cells' ability to explore their genomic landscape and thus reduce their ability to adapt and evade chemotherapies. In some embodiments, the CPT-chemotherapy (or CPT-immunotherapy) combination significantly enhances the efficacy of existing treatments (e.g., chemotherapies and/or immunotherapies).

CPT agents regulate chromatin nanoenvironment toward a normalized, constrained, and less-adaptive state. The principles of the CPT strategy are based on several observations. First, the ability of cancer cells to sample their global genomic (transcriptional) landscape is a critical cause of chemo-resistance (Ref. 10c; incorporated by reference in its entirety). Second, these aspects are regulated by supra-nucleosomal chromatin folding (Refs. 13c-16c; incorporated by reference in their entireties). Results demonstrate that increased heterogeneity of chromatin nanoenvironment at the supra-nucleosomal scales allows cancerous cells to explore GGL and change their global patterns of gene expression (ref. 10c; incorporated by reference in its entirety). Third, experiments were conducted during development of embodiments herein to develop a platform of new nanoimaging technologies which quantitatively interrogate spatio-temporal changes in the chromatin nanoenvironment (Refs 15c-16c; incorporated by reference in their entireties) and quickly test compounds in regards to their ability to normalize chromatin heterogeneity and thus identify CPT agents. Fourth, a number of CPT agents have been identified among existing drugs that have been traditionally used for non-cancer indications. CPT compounds reduce the heterogeneity of chromatin nanoenvironment and thus reduce cancer cells' ability to adapt to chemotherapies. CPT in combination with chemotherapy drugs have been shown to achieve 100% cancer cell death in multiple cancer lines in vitro.

Chemotherapy rarely leads to complete remission of most solid cancers. Immunotherapy has the potential to revolutionize cancer treatment but for most solid tumors the remission rate is still low. Five-year survival for unresectable cancers is typically <10%; the curation rate is even lower (Ref 17c; incorporated by reference in its entirety). A new targeted- or immuno-therapy drug that increases progression-free survival by a few months is heralded as a major breakthrough. Why do so many anticancer therapeutics fail? Anticancer drugs come in many varieties including antimetabolites, topoisomerase inhibitors, alkylators, anti-tumor antibiotics, mitotic inhibitors, corticosteroids, hormones and their antagonists, biologically targeted agents, and immuno-targeted agents (Ref. 2c; incorporated by reference in its entirety). Most of the existing anti-cancer drugs are cytotoxic. This cytotoxicity is induced through a variety of pathways including DNA damage (e.g. intercalating agents), the disruption of other cellular structures (e.g. damage to microtubules), the activation of the immune system attacking the tumor cells, etc. A single major reason why anti-cancer drugs fail is because cancer cells eventually develop resistance to almost all chemotherapeutic drugs via a variety of mechanisms including reduced drug accumulation and/or increased drug export, alterations in drug targets and signaling transduction molecules, repair of drug-induced DNA damage, evasion of apoptosis, etc. (Ref. 2c; incorporated by reference in its entirety).

Tumor heterogeneity is a critical factor in primary drug resistance (Refs. 5c-6c; incorporated by reference in their entireties) (e.g. even if some tumor clones may succumb to the therapy, other clones may already be resistant to the drug and thus will continue to proliferate with the therapy essentially removing the competition) as well as the emergence of new drug-resistant clones (acquired resistance) (Ref. 9c; incorporated by reference in its entirety)). New gene mutations are not always necessary for drug resistance, and a change in the expression of existing genes may suffice (Ref 8; incorporated by reference in its entirety). Transient transcriptional states play an important role in chemoresistance: although distinct from the gene mutation-dependent adaptation, the transcriptional heterogeneity-dependent adaptation indirectly facilitates gene mutations by allowing cells to survive the adverse stimuli long enough for the mutations to occur (Ref. 7c; incorporated by reference in its entirety). This transcriptional heterogeneity is manifested both in cells being able to dynamically sample different transcriptional states of multiple genes and in the intercellular diversity of expression (e.g., any given gene is expressed at different levels across a cell population) (Ref. 7c; incorporated by reference in its entirety). Cancer cells have a remarkable capacity to adapt by dynamically changing their global gene expression patterns (Ref. 10c; incorporated by reference in its entirety). Chemoresistance is facilitated by a link between the overall rate of transcription, which as data indicates increases in GGL exploration, and clonal evolution: genes that are transcribed at a higher rate have a greater likelihood of being mutated (Ref 18c; incorporated by reference in its entirety).

CPT leverages supra-nucleosomal chromatin folding as a key regulator of non-replicative cell adaptability through the exploration of the transcriptional landscape (Refs. 10c-12c; incorporated by reference in their entireties). The function of CPT agents is to reduce the ability of cancer cells to adapt and develop drug resistance, thus improving the efficacy of existing therapies. CPT agents manipulate the chromatin structure, reduce cancer cells' ability to explore GGL, and have profound synergistic anticancer properties, particularly when paired with standard chemotherapy and/or immunotherapy agents. Clinically, CPTs increase the effectiveness of conventional chemotherapies and/or immunotherapies, for example, at a much lower dose (e.g., decreased toxicity). CPTs are useful as a combination therapy to prevent development of tumor heterogeneity and resistance to most existing therapies including chemo-, immuno-, and targeted-therapies.

The exploration of the GGL has critical implications for early carcinogenesis and chemotherapy. Expansion of the population heterogeneity stabilizes otherwise deleterious gene mutations, and potentiates tumor formation by increasing the likelihood of finding stable negative states. Furthermore, increased exploration of the GGL aids in the development of new traits unique to tumors, such as angiogenic induction or stabilization of abnormal metabolism. This has important ramifications for chemotherapy. The current strategy behind most existing anti-cancer chemotherapies is to kill as many cancer cells as possible while preserving non-cancer cells, to the extent possible. Consider a highly potent drug that kills 99.9% of cancer cells. After therapy, ˜10⁵ cancer cells will still survive per each gram of the original tumor (ref 23a; incorporated by reference in its entirety). However, clonal expansion alone does not characterize the distribution of evasive mechanisms found within the surviving cells. The heterogeneity of the chromatin nanoenvironment helps cells to explore a larger genomic information space; coupled with a strong selective pressure (e.g. a chemotherapeutic agent), this leads to the emergence of new drug-resistant clones due to cells finding new evasive mechanisms during treatment. This is reminiscent of antibiotic treatment of bacterial infections: bacteria evolve at the timescale of treatment, which eventually leads to the emergence of drug-resistant organisms. Thus, in some embodiments, CPTs, by reducing chromatin heterogeneity, provides (1) in the development of new traits unique to tumors, and/or (2) inhibition of resistance/evasion of treaments by existing cancers/tumors. In some embodiments, CPTs and methods of use thereof inhibit cancer cells' ability to evolve and develop drug resistance, thus improving the efficacy of other (e.g., existing) therapies/therapeutics. In some embodiments, CPTs and methods of use thereof inhibit the formation of cancers/tumors, for example, in subjects with risk factors for the development of cancer (e.g., previous cancer, genetic susceptibility, exposure to mutagen, etc.).

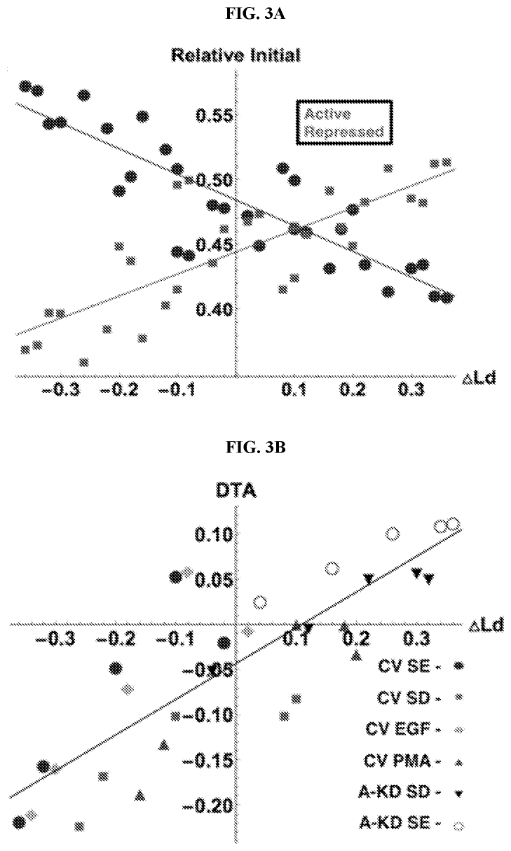

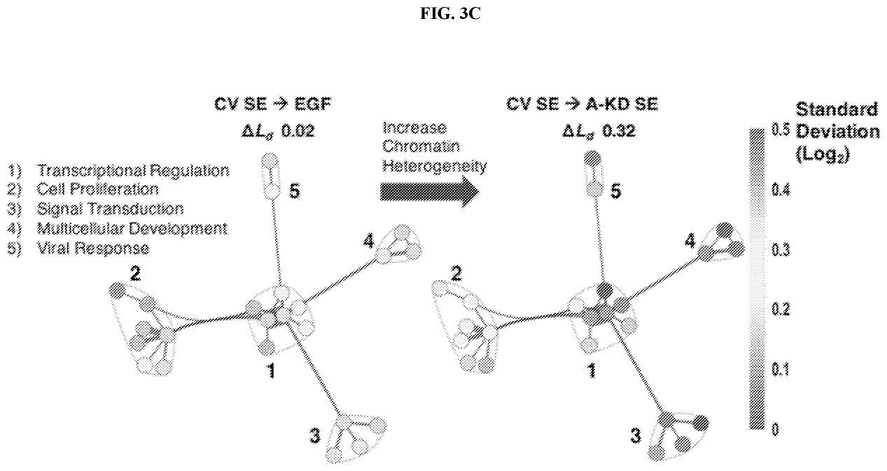

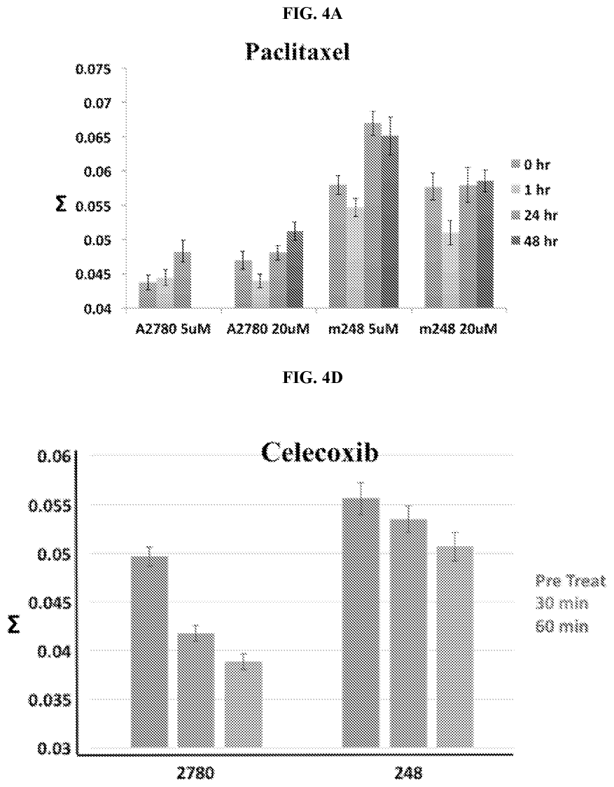

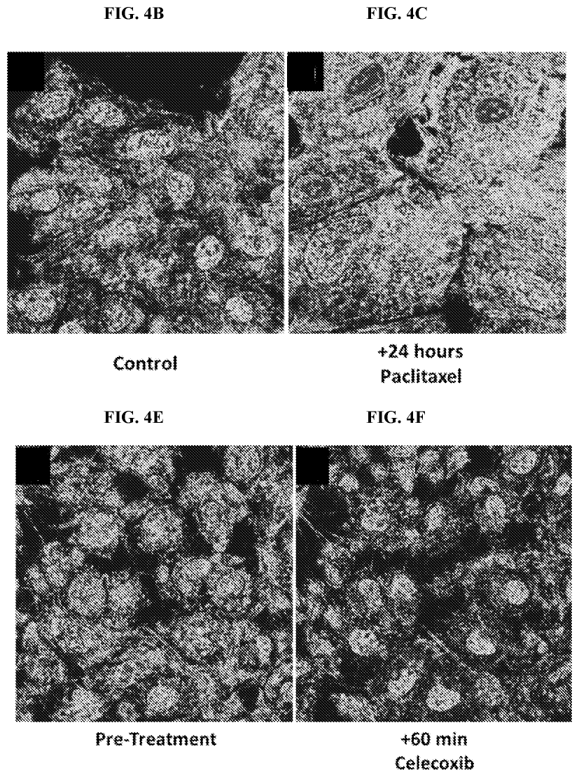

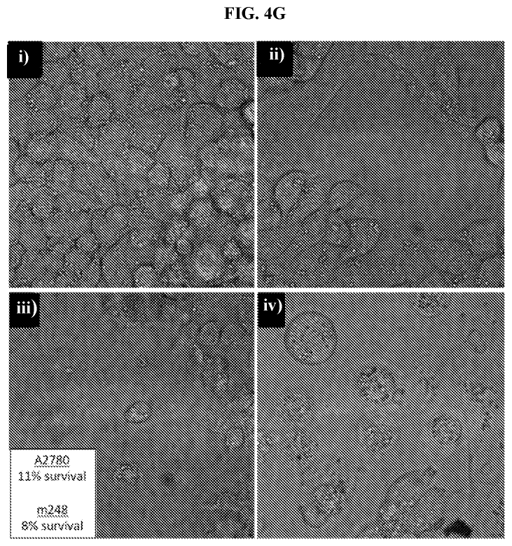

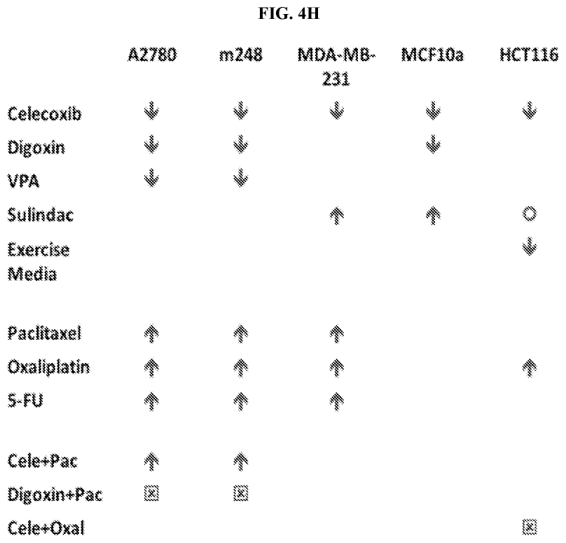

In some embodiments, CPTs and methods of use herein do not target the specific drivers of tumor formation or treatment evasion; rather, they limit the extent of genomic exploration by targeting variations in the physical structure of chromatin. A CPT approach limits the degrees of freedom present within chromatin by regulating the overall physical structure (e.g., targeting topological variations). Since, variations in chromatin structure from cell-to-cell allows cells to search for new mechanisms that aid in survival at low energetic cost, CPTs inhibit this search. Experiments conducted during development of embodiments herein indicate a correlation between heterogeneity of chromatin organization (e.g., fractal dimension) and the heterogeneity of gene expression for critical processes, including proliferation and apoptosis (FIG. 3). Increased chromatin heterogeneity has been consistently observed preceding the development of tumors in both human and animal models of carcinogenesis (refs. 25-28; incorporated by reference in their entireties). Likewise, theoretical modeling and experimental results have shown that changes in the physical environment independently modulate transcription (refs. 29-30; incorporated by reference in their entireties). Experiments conducted during development of embodiments herein exploring the effects of chemotherapeutic agents on chromatin topology have consistently found increases in the fractal dimension of chromatin across multiple tumor models (e.g., colon, breast, ovarian, cervical cancer) in the cells that evade treatment, independent of the chemotherapeutic agent (e.g., oxilaplatin, 5-FU, paclitaxel, docetaxel, and gemcitabine) (FIG. 4).

The physical transformation of chromatin has a significant role in tumor formation and chemoresistance, independent of effects mediated by epigenetic chemical modifications. Therefore, physiochemical regulators, that control the overall heterogeneity of chromatin structure, for example, by targeting metal-ion homeostasis or Ψₘ, provide therapeutic and/or prophylactic therapy for cancers.

In some embodiments, CPTs complement existing strategies by decreasing the cumulative adaptive potential of tumor cells. In such embodiments, an adjuvant CPT decreases the probability of emergence of secondary proliferative and evasive mechanisms through restriction of the possible configurations of chromatin. By acting on the overall physical structure, CPTs restrict the global sampling capacities of cells to reduce the combinatorial dimensions of evasion (FIG. 2).

In certain embodiments, CPTs provide a prophylactic approach, for example, for subjects with high-risk mutations, by preventing accumulated sampling in addition to the known drivers of tumor formation. Prophylactic CPTs, or methods of using CPTs prophylactically, restrict the accumulation of adaptions before, during, between, or after courses of conventional treatments.

In some embodiments, CPTs prevent sampling of different states during stress, thereby considerably reducing the population of surviving cancer cells to those that previously acquired a favorable initial evasive state.

In some embodiments, a CPT is a small molecule, peptide, antibody, etc. that reduces chromatin heterogeneity across a population of cells. In some embodiments, CPTs decrease the fractal dimension, and normalize the chromatin nanoenvironment.

In some embodiments, CPTs are provided herein.

In some embodiments, systems and methods are provided for the identification and characterization of CPTs. In some embodiments, CPT identification is performed using high-throughput live-cell Partial Wave Spectroscopic Microscopy (HTLC-PWS). Partial wave spectroscopic microscopy is a nanoscale sensitive imaging modality that quantifies the underlying physical structure within cells. This technique allows rapid identification of agents (e.g., drugs) that regulate the overall nanostructure of chromatin (e.g., fractal dimension). Large changes in fractal dimension of chromatin structure result in increased capacity of cells to more thoroughly sample the GGL. Experiments conducted during development of embodiments herein demonstrate the structure-function relationship between chromatin structure and gene expression. Measuring alterations in the physical topology of chromatin allows insight into the underlying molecular transformations occurring in gene expression (FIG. 3). Consequently, drugs that modulate the physical topology of chromatin are screened by LC-PWS microscopy in real-time. Experiments conducted during development of embodiments herein have found that Ψₘ-depleting agents, such as carbonyl cyanide m-chlorophenyl hydrazone (CCCP), potassium ionophores (e.g., digoxin and valinomycin), and non-steroidal anti-inflammatory drugs (NSAIDs) (e.g., celecoxib and aspirin), are potent regulators of chromatin topology, changing the overall nanoarchitecture within minutes of treatment.

In some embodiments, CPTs are identified based on their physio-chemical action (e.g., decrease in the fractal structure (e.g., normalization) of the chromatin nano-environment. Exemplary categories of agents that may be searched for CPTs include, but are not limited to: mTOR-regulators (e.g. rapalogs), metabolic modifiers (e.g. CCCP, oligomycin), NSAIDs (e.g. sulindac, celecoxib), iono-modulatory agents (e.g. digoxin/oubain, valinomycin, ionomycin), and multi-pathway agents (e.g. ACE inhibitors, metformin, aspirin, β-blockers). In some embodiments, the efficacy of these compounds is tested by introducing the candidate compounds individually and monitoring the real-time changes in chromatin nanostructure of chemoresistant, chemosensitive, and non-neoplastic primary cell lines using LC-PWS microscopy. In some embodiments, a large number (e.g., 10², 10³, 10⁴, 10⁵, 10⁶, and ranges therebetween) of candidate agents are screened using HTLC-PWS.

In some embodiments, candidate CPTs satisfy one or more of the following criteria: (i) reduce chromatin heterogeneity in cancer cells without significant effects on normal cells, (ii) reduce GGL sampling capacity of cells (e.g., as assayed by, for example, PWS, gene expression analyses, etc.), (iii) increase cellular response to chemotherapeutic agents, etc. In some embodiments, screening for CPTs follows a recursive method, for example: (1) study dose response effect on chromatin heterogeneity, (2) measure the variability of gene expression including expression of resistance markers, (3) demonstrate increased efficacy when paired with existing chemotherapeutic.

In some embodiments, screening is performed to identify CPTs that work as adjuvants for existing chemotherapeutics. In some embodiments, screening is performed to identify CPTs that reduce the risk of tumor formation as low dose prophylactics.

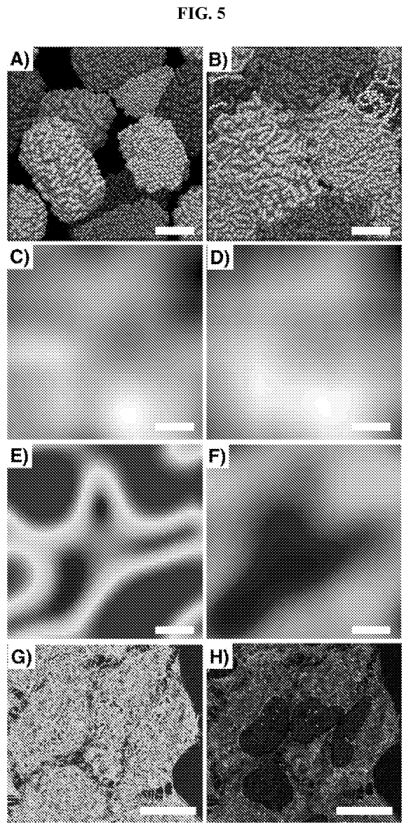

In some embodiments, animal models of tumor formation are treated with CPTs (e.g., low-dose) and the rate of tumor formation is measured/monitored. For example, changes into the chromatin structure within the affected tissue are measured for control (e.g., vector-treated), CPT-treated, and non-treated test subjects. As with the above approach for adjuvants, in some embodiments, in situ sequencing is performed in addition to, or instead of, LC-PWS microscopy to measure variability in gene expression in addition to paired histological analysis of tumor progression (FIG. 5).

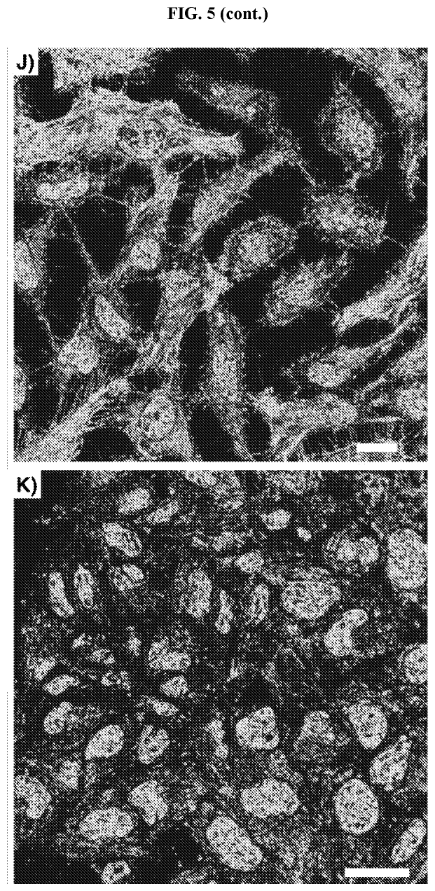

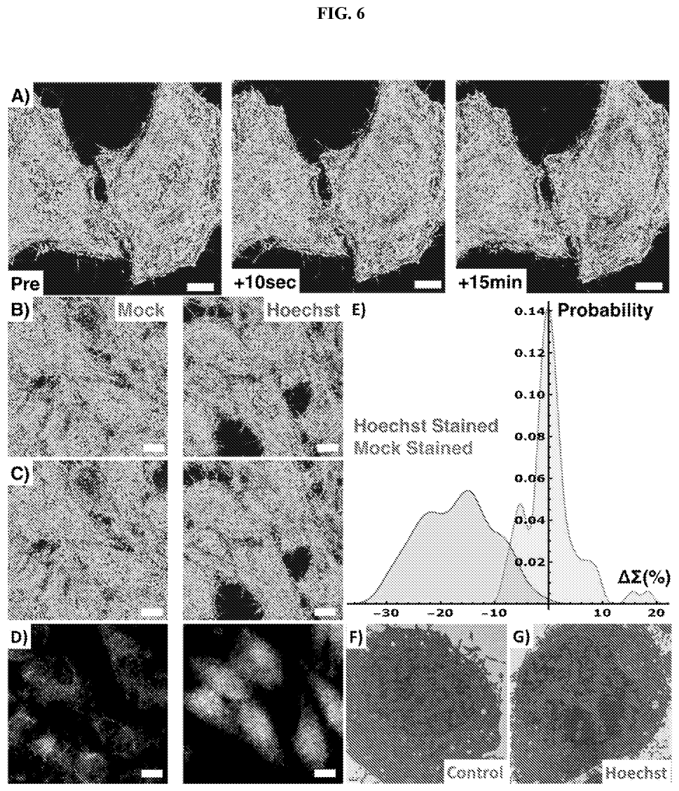

In some embodiments, high throughput LC-PWS microscopy is utilized for real-time study of patient-derived cells and their response to CPTs and standard chemotherapeutics. High throughput LC-PWS has the capacity to analyze nanoscale structure and dynamics, as well as acquire molecular specific information using fluorescent based strategies. In some embodiments, personalized CPTs are developed using an automated multi-well plate acquisition system to acquire treatment response behavior for cells isolated from patient's primary and metastatic tumor biopsies. Using the automated scanning, LC-PWS collects data on nanoscale structure and dynamics, phase contrast, cell viability measurements, organelle function, etc. As the tissue is unmodified by the LC-PWS measurements, in some embodiments it is subsequently utilized for transcriptome, genome, metabolome, and/or proteomic analysis. Using techniques such as immunofluorescence (IF), fluorescence in situ hybridization (FISH), and in situ sequencing, this multimodal approach determines the efficacy of existing chemotherapeutics and determines secondary treatment options (FIG. 6). As measurements are automated, the protocols are optimized with case-by-case selection of therapies determined by the combination of CPT responsiveness, existing resistance phenotypes, and chemotherapeutic efficacy. In this manner, patients are provided idealized combination therapies.

Other technologies that find use in the screening of CPTs include, for example, systems and methods capable of measuring either molecular transformations in higher order chromatin or physical changes in chromatin topology. These techniques include, but are not limited to: electron microscopy, super resolution microscopy (e.g., STED, PALM/STORM, SIM, etc.), chromatin capture methods (e.g., HI-C, 5C, 3C, etc.), chromatin immunoprecipitation methods (CHIP-Seq, MNase-Seq, etc.).

In some embodiments, the CPT-screening methods described herein have identified a number of agents which act on the level of chromatin topology. The compounds that modulate the structure of chromatin in live cells, for example, by reducing Σ, increase chemotherapeutic efficacy, even up to over 90% elimination (Table 1). Additional experiments conducted during development of embodiments herein have demonstrated that aspirin, celecoxib, digoxin, valinomycin, CCCP, and exercise media all act as global regulators of chromatin heterogeneity.

TABLE 1

| | Chemo | CPT | % Inhibition |
|---|---|---|---|
| Ovarian | | | |
| A2780 | Paclitaxel | | 21 |
| | | Celecoxib | 0 |
| | Paclitaxel | Celecoxib | 96 |
| | Paclitaxel | Digoxin | 100 |
| A2780.m248 | Paclitaxel | | 57 |
| | | Celecoxib | 0 |
| | Paclitaxel | Celecoxib | 94 |
| | Paclitaxel | Digoxin | 100 |
| Sarcoma | | | |
| MES-SA | Docetaxel | | 65 |
| | Docetaxel | Digoxin | 80 |
| | Docetaxel | Celecoxib | 100 |
| MES-SA MX2 | Docetaxel | | 79 |
| | Docetaxel | Digoxin | 85 |
| | Docetaxel | Celecoxib | too |
| Breast | | | |
| MDA-MB-231 | Paclitaxel | | 50 |
| | Paclitaxel | Celecoxib | 86 |
| | Paclitaxel | Digoxin | 79 |

The organization of chromatin is a regulator of molecular processes including transcription, replication, and DNA repair. The structures within chromatin that regulate these processes span from the nucleosomal (10 nm) to the chromosomal (>200 nm) levels.

In some embodiments, provided herein are biophysical techniques for the measurement (e.g., quantitatively, qualitatively) of chromatin heterogeneity in a population of cells. Suitable techniques include partial wave spectroscopic microscopy, super-resolution fluorescence microscopy (SRM), etc. In some embodiments, live-cell PWS is employed. In some embodiments, high-throughput live-cell Partial Wave Spectroscopic Microscopy (HTLC-PWS) is employed.

Partial wave spectroscopic (PWS) microscopy is a quantitative imaging technique with sensitivity to macromolecular organization between 20-200 nm, which has shown that transformation of chromatin at these length scales is a fundamental event during carcinogenesis. As the dynamics of chromatin play a critical regulator role in cellular function, experiments were conducted during development of embodiments herein to develop live-cell imaging techniques that probe the real-time temporal behavior of chromatin nanoarchitecture. Live cell PWS techniques were developed which allow high-throughput, label-free study of the causal relationship between nanoscale organization and molecular function in real-time. In this work, live cell PWS is employed to study the change in chromatin structure due to DNA damage and expand on the link between metabolic function and the structure of higher-order chromatin. In some embodiments, techniques allow the monitoring of temporal changes to chromatin during DNA damaging conditions (e.g., UV light exposure, DNA damaging agents, etc.). Experiments demonstrate that damage may be induced to chromatin within seconds, and demonstrate a direct link between higher-order chromatin structure and mitochondrial membrane potential. Since biological function is tightly paired with structure, live cell PWS provides a useful tool to study the nanoscale structure-function relationship in live cells.

Every cellular and extracellular structure has a complex nanoscale organization ranging from individual macromolecules that are a few nanometers in size (e.g. protein, DNA) to macromolecular assemblies that are tens to hundreds of nanometers in size (e.g. cell membranes, higher-order chromatin structure, cytoskeleton, and extracellular matrix fibers). A major scientific challenge is to understand these macromolecular structures, specifically their function and interactions in structurally and dynamically complex living cellular systems. To meet these goals, the ideal live cell imaging technology would satisfy five key requirements: (1) nanoscale sensitivity (<200 nm), (2) label-free (3) non-perturbing (4) quantitative, (5) high-throughput, and (6) molecularly informative.

Previous approaches are unable to meet all these criteria alone. The breakthroughs in super-resolution fluorescence microscopy (SRM) have enabled new imaging technologies capable of providing unprecedented molecular identification at the highest resolutions currently available in live cells, but require the use of exogenous fluorophores to visualize macromolecular structures (refs. 1b-3b; incorporated by reference in their entireties). For some applications, these labels are indispensable to achieve molecular specificity. However, there are significant drawbacks to the exclusive use of molecular labels for studies of cellular structure and function. Exclusively label-based SRM approaches are limited by the number of possible imaging channels, the high label-densities required, the high light intensities utilized during imaging, and by the introduction of possible artifacts due to the labels themselves, especially at the high densities required for nanoscale resolution (refs. 4b, 5b; incorporated by reference in their entireties). In the study of macromolecular organization, current imaging approaches have significant drawbacks as macromolecular structures are often composed of dozens to hundreds of distinct molecules and often include different subtypes such as lipids, proteins, nucleic acids, and carbohydrates, some of which are difficult to directly label. Due to these limitations, phase contrast microscopy is still the most widely used label-free imaging modality for live cell experiments. While this technique is fast and improves contrast to visualize live cells, its diffraction-limited resolution cannot provide any insights into the macromolecular nano-architecture. As such, currently no label-free optical technique exists to measure the nano-architectural properties of live cells below the diffraction limit.

One prominent area of biological research with a demonstrated need for label-free, nanoscale sensitive imaging is the investigation of the structure-function relationship of chromatin. Chromatin organization (which is comprised of DNA, histones, and hundreds of other conjugated proteins and small molecules such as RNA) involves a hierarchy of length scales ranging from a few tens of nanometers in nucleosomes to hundreds of nanometers for chromosomal territories (refs. 6b, 7b; incorporated by reference in their entireties). The physical nanostructure of chromatin is regulated by numerous molecular factors, including the primary DNA sequence composition, differential methylation patterns, histone modifications, polycomb and cohesion protein complexes, RNA and DNA polymerases, long non-coding RNA, etc, and non-molecular factors, such as crowding, ionic conditions, and pH. Due to this complexity and limitations in existing optical techniques that can rapidly sample information below 200 nm, little is known about the higher-order chromatin structure between these length scales or their dynamics in live cells. Results from fixed cell imaging techniques, such as electron microscopy or SRM, have shown that chromatin between 20-200 nm is first organized into poly-nucleosomal 10 nanometer fibers, and in certain conditions, these fibers have been shown to assemble into 30 nm clusters (refs. 8b-10b; incorporated by reference in their entireties), although the existence of the 30 nm fiber is a subject of an active debate. At length scales between 100-200 nm, recent work using SRM has shown a power-law relation in the organization of chromatin, with domains of highly-dense, inactive chromatin localizing within a few-hundred nanometers of transcriptionally active sites (ref. 11; incorporated by reference in its entirety). In conjunction, molecular techniques such as chromosomal capture methods (3C and Hi-C) have shown that the higher-order organization of chromatin above single nucleosomes and below the structure of chromosomal territories follows this same power-law fractal organization. These methods have shown that topology of this higher order organization is correlated with the regulation of gene transcription (refs. 12-14; incorporated by reference in their entireties) and capable of evolving rapidly under stress conditions to globally regulate the expression of genes (ref. 15; incorporated by reference in its entirety). Critically, these observed changes in chromatin structure have recently been linked to the regulation of genes often implicated in oncogenesis (ref. 16b; incorporated by reference in its entirety).

Chromatin topology at all length scales are a critical determinant of tumor formation, aggressiveness, and chemoresistance. One of the primary features of tumorigenesis is a shift in the fractal physical organization of chromatin, correlating both with the formation of tumors and their invasiveness. PWS microscopy allows examination of the intracellular organization concealed by the diffraction limit with length scale sensitivity in the range of 20-200 nm, the range at which existing label-free live cell imaging techniques are deficient, due to the relationship between the nanoscale spatial variations of macromolecular density and the resulting variance in the spectrum of backscattered light (refs 17b, 18b; incorporated by reference in their entireties). Sub-diffractional structures are detected by analyzing the PWS spectrum of elastically scattered light to provide quantitative contrast.

Experiments were conducted during development of embodiments herein to create a label-free live cell microscopy method based on interference principles used in PWS cytology, thereby creating a tool to directly study the dynamic nanoscale topology of live cells, with a specific focus on studying real-time changes in chromatin organization. The HT-LC-PWS techniques described herein: (1) provide nanoscale sensitivity to structures between 20-200 nm, (2) use label-free contrast to capture nanoscopic information, (3) are non-perturbing to biological samples by using low power illumination and label-free contrast, (4) quantify the cellular nano-architecture, and (5) rapidly capture the temporal evolution of nanoscale structures, providing contrast in multiple cells in seconds. Live cell PWS is its unique ability to work in conjunction with existing label-based technologies to provide the structural context for molecular interactions, thereby greatly expanding the understanding of the molecular behavior in live cells (ref 19; incorporated by reference in its entirety). Using this approach, experiments conducted during development of embodiments herein demonstrate the ability to measure the nano-architecture in live cells in seconds. Specifically, techniques herein explore changes to the cellular nano-architecture due to UV light exposure, show that live cell DNA binding dyes transform chromatin within seconds, and demonstrate a direct link between higher-order chromatin structure and mitochondrial membrane potential. In some embodiments, PWS, and HT-PWS systems, devices, and techniques described in, for example, U.S. Pub. No. 2015/0292036; U.S. Pub. No. 2015/0029326; U.S. Pub. No. 2014/0302514; U.S. Pub. No. 2012/0214880; U.S. Pub. No. 2008/0278713; and U.S. Pub. No. 2008/0180664 (each of which are herein incorporated by reference in their entireties), find use in embodiments herein.

In some embodiments, provided herein are chromatin protective therapeutics, methods of screening and validating test agents as CPTs (e.g., as a chemotherapeutic, as a cancer prevention, as an adjunct to other chemotherapeutics, etc.), systems and devices for monitoring chromatin heterogeneity (e.g., high throughput, live cell, etc.), methods of treat of treating a subject suffering from cancer, methods of preventing cancer and/or tumor formation, etc.

Cells evade/respond to environmental or other stresses through sampling of available genomic information. Chromatin heterogeneity allows for a population of cells to maximize the available genomic information and increases the likelihood of successfully evading/responding to the stress. Chromatin heterogeneity also maximizes the likelihood that a population of cells will become cancerous, form a tumor, spread or metastasize, and/or develop resistance to therapeutics. As such therapies and therapeutics that reduce chromatin heterogeneity are useful in the treatment and/or prevention of cancers, either as stand-alone treatment, or as co-therapeutics with other anti-cancer treatments/therapies.

Experiments conducted during development of embodiments herein have demonstrated that live-cell PWS (LC-PWS) and/or high-throughput live-cell PWS (HT-LC-PWS) is an effective and efficient screen for identifying agents capable of reducing chromatin heterogeneity in a cell population, and therefore in limiting the pathways that lead to caner/tumor formation, spread, resistance, etc. In some embodiments, methods are provided herein of testing compounds, peptides, antibodies, etc. for activity in inhibiting and/or reducing chromatin heterogeneity. In some embodiments, agents capable of reducing chromatin heterogeneity in a cell population are chromatin protective therapeutics (CPTs) and find use in the treatment or prevention of cancer (e.g., all cancers, a specific subset of cancers (e.g., solid tumor cancers), a specific type of cancer (e.g., breast cancer, thyroid cancer, brain cancer, liver cancer, lung cancer, colon cancer, prostate cancer, etc.). In some embodiments, methods are provided for identifying and/or designing CPTs, using LC-PWS to identify agents that inhibit/reduce chromatin heterogeneity. In some embodiments, HT-LC-PWS allows screening of large numbers of agents and/or modifications to agents of interest.

In some embodiments, compositions (e.g., pharmaceutical compositions) comprising one or more CPTs are provided. In some embodiments, a CPT is generally applicable to all cell types, cancer types, etc. In other embodiments, a CPT is particularly applicants to one or more cell types, tissue types, routes of administration, cancer types, etc.

Exemplary CPTs have been identified in experiments conducted during development of embodiments herein, such as celcoxib:

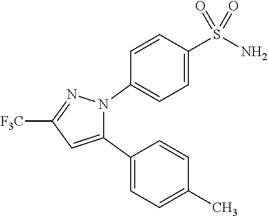 [SMILES: Cc1ccc(-c2cc(C(F)(F)F)nn2-c2ccc(S(N)(=O)=O)cc2)cc1]

and digoxin:

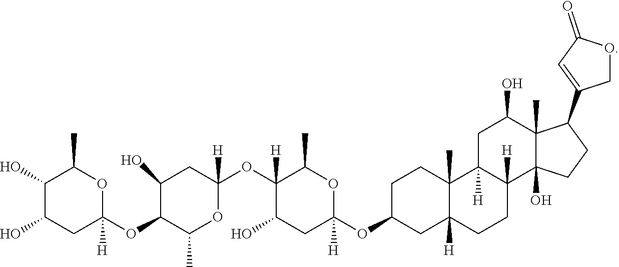

In some embodiments, the screening techniques described herein (e.g., using LC-PWS) are used to identify modifications to the above compounds that are particularly useful as CPTs. Other agents identified using the screening techniques described herein are within the scope of embodiments herein.

In some embodiments, a CPT is administered systemically to a subject (e.g., intravenously, transdermally, orally, etc.). In some embodiments, a CPT is administered or delivered to a specific organ, tissue, cell type (e.g., cancer cell, tumor cell), location (e.g., tumor), etc.

In some embodiments, a CPT is administered as a direct therapeutic agent or prophylactic to treat or prevent cancer. In some embodiments, a CPT is co-administered with an additional therapy (e.g., radiation, surgery, etc.) or therapeutic (e.g., chemotherapeutic). In some embodiments, a CPT is provided as supplement to one or more other therapies or therapeutics. In some embodiments, a CPT increases the efficacy of another therapy or therapeutic. In some embodiments, a CPT prevents immune evasion, development of resistance to a therapeutic, metastasis, or other mechanisms by which cancer spreads and/or evades treatment (e.g., in response to treatment). In some embodiments, the benefit derived from a primary treatment is enhanced by co-administration with a CPT.

In some embodiments, administration of a CPT is sufficient on its own to treat or prevent cancer and/or tumor growth. However, in other embodiments, CPTs are co-administered with one or more other cancer therapies. In some embodiments, CPT treats cancer by a mechanism independent of one or more additional cancer treatments. In some embodiments, the CPT enhances the effectiveness of the other cancer treatment(s). Embodiments herein are not limited by the types of cancer treatments (e.g., surgery, radiation, immunotherapy, chemotherapeutic, etc.) unless specifically noted.

In some embodiments, co-administration with a CPT allows the other therapeutic to be administered at a subtherapeutic dose. In some embodiments, administration at a subtherapeutic dose reduces the likelihood of resistance developing to the treatment, reduces side effects, prolongs the amount of time a subject may receive the treatment, etc.

Many chemotherapeutics are presently known in the art and can be used in combination with (e.g., co-administered with) the CPTs described herein and identified through screening. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Embodiments further relate to using CPTs in combination with (e.g., co-administered with) radiation therapy for inhibiting abnormal cell growth or treating a hyperproliferative disorder. Techniques for administering radiation therapy are known in the art, and these techniques can be used in the combination therapy described herein. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

In some embodiments, CPTs are used in combination with (e.g., co-administered with) an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with a compound of the invention and pharmaceutical compositions described herein. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606, 046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, and MMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATGS (which are implicated in autophagy), may also be used.

In some embodiments, CPTs are used in combination with (e.g., co-administered with) an immunotherapy treatment. Suitable immunotherapies may include, but are not limited to: cell-based therapies (e.g., dendritic cell or T cell therapy, etc.), monoclonal antibody (mAb) therapy (e.g., naked mAbs, conjugated mAbs), cytokine therapy (e.g., interferons, interleukins, etc.), adjuvant treatment (e.g., polysaccharide-K), etc.

In some embodiments, immunotherapeutic cancer treatment encompasses blockade of immune-inhibitory receptors, for example using monoclonal antibodies (mAbs) against CTLA-4 and PD-1/PD-L1 (Wolchok, J. D. et al. The New England Journal of Medicine 369, 122-133 (2013); Topalian, S. L. et al. Journal of clinical oncology 32, 1020-1030 (2014); Topalian, S. L. et al. The New England journal of medicine 366, 2443-2454 (2012); Hodi, F. S. et al. The New England journal of medicine 363, 711-723 (2010); herein incorporated by reference in their entireties).

In some embodiments, the immunotherapy includes the administration of an immune checkpoint inhibitor. Immune Checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012 and STI-A1010. In some embodiments, the immune checkpoint inhibitor may be administered via injection (e.g., intravenously, intratumorally, subcutaneously, or into lymph nodes), but may also be administered orally, topically, or via aerosol.

Examples of antibodies that may find use in the compositions and methods disclosed herein (e.g., for co-administration with a CPT), particularly for use in immunotherapies (but not so limited) include, but are not limited, to antibodies such as trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb): Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (@(17-1A)) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym). Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab, or ImmuRAIT-CEA.

In some embodiments, an immunotherapy, utilized as a co-therapy with the CPTs described herein, directly or indirectly targets one of more of: a regulatory T cell, myeloid suppressor cell, or dendritic cell. In another aspect, an immunotherapy specifically targets one of the following molecules: CD4; CD25 (IL-2α receptor; IL-2αR); cytotoxic T-lymphocyte antigen-4 (CTLA-4; CD152); Interleukin-10 (IL-10); Transforming growth factor-beta receptor (TGF-βR); Transforming growth factor-beta (TGF-β); Programmed Death-1 (PD-1); Programmed death-1 ligand (PD-L1 or PD-L2); Receptor activator of nuclear factor-KB (RANK); Receptor activator of nuclear factor-κB (RANK) ligand (RANKL); LAG-3; glucocorticoid-induced tumor necrosis factor receptor family-related gene (GITR; TNFRSF18); or Interleukin-4 receptor (IL-4R). In some embodiments, the immunotherapy acts as an agonist that increases the function of the targeted molecule. In other embodiments, the immunotherapy is an antagonist that inhibits the function of the targeted molecule.

In some embodiments, an immunotherapy, utilized as a co-therapy with CPTs described herein, directly or indirectly targets one of more of a specific cytokine, cytokine receptor, co-stimulatory molecule, co-inhibitory molecule, or immunomodulatory receptor that modulates the immune system. In another aspect, one of the following molecules are targeted by co-treatment with microflora modulation: tumor necrosis factor (TNF) superfamily; tumor necrosis factor-α (TNF-α); tumor necrosis factor receptor (TNFR) superfamily; Interleukin-12 (IL-12); IL-12 receptor; 4-1BB (CD137); 4-1BB ligand (4-1BBL; CD137L); OX40 (CD134; TNR4); OX40 ligand (OX40L; CD40; CD40 ligand (CD40L); CTLA-4; Programmed death-1 (PD-1); PD-1 ligand I (PD-L1: B7-H1); or PD-1 ligand 2 (PD-L2; B7-DC); B7 family; B7-1 (CD80); B7-2 (CD86); B7-H3; B7-H4; GITR/AITR: GITRL/AITRL; BTLA; CD70; CD27; LIGHT; HVEM: Toll-like receptor (TLR) (TLR 1, 2, 3, 4, 5, 6, 7, 8, 9, 10).

In some embodiments, medicaments which are administered in conjunction with the compounds described herein include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatories, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

Other exemplary therapeutic agents useful for a combination therapy include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

In some embodiments, therapeutic agents for co-administration with CPTs also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, Mycobacterium avium complex disease, and leprosy, antifungal agents, antiviral agents including nonretroviral agents and antiretroviral agents.

Examples of therapeutic antibodies that can be combined with CPTsinclude but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

Further therapeutic agents that can be combined with CPTs are found in Goodman and Gilman's “The Pharmacological Basis of Therapeutics” Tenth Edition edited by Hardman, Limbird and Gilman or the Physician's Desk Reference, both of which are incorporated herein by reference in their entirety.

In some embodiments, CPTs find use in combination with the agents disclosed above or other suitable agents, depending on the condition being treated. Hence, in some embodiments the one or more compounds of the invention will be co-administered with other agents as described above. When used in combination therapy, CPTS and/or co-administered agents are administered simultaneously or separately. This administration in combination can include simultaneous administration of the two agents in the same dosage form, simultaneous administration in separate dosage forms, and separate administration. That is, a CPT and any of the agents described above can be formulated together in the same dosage form and administered simultaneously. Alternatively, a CPT and any of the agents described above are simultaneously administered, wherein both the agents are present in separate formulations. In another alternative, a CPT is administered just followed by and any of the agents described above, or vice versa. In some embodiments of the separate administration protocol, a CPT and any of the agents described above are administered a few minutes apart, or a few hours apart, or a few days apart.

In some embodiments, any of the aforementioned agents may find use in the screening embodiments described herein. For example, one or more of the above agents may be tested for CPT activity using LC-PWS. In some embodiments, to the extent that any of the above agents find use as a CPT (e.g., to the extent that they inhibit or reduce chromatin heterogeneity), such agents find us in the therapeutic/preventative methods and compositions (e.g., pharmaceutical compositions) described herein.

In some embodiments, CPTs find use in the treatment of prevention of cancer. Non-limiting examples of cancers that may be treated with the compositions and methods described herein include, but are not limited to: cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is a melanoma (e.g., metastatic malignant melanoma), renal cancer (e.g. clear cell carcinoma), prostate cancer (e.g. hormone refractory prostate adenocarcinoma), pancreatic cancer (e.g., adenocarcinoma), breast cancer, colon cancer, gallbladder cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Some embodiments described herein are particularly useful for the treatment of cancers/tumors that do not otherwise respond to one or more other therapeutic approaches (e.g., chemotherapeutic approaches, immunotherapeutic approaches, etc.).

### EXPERIMENTAL

### Example 1

### Live Cell PWS

Experiments conducted during development of embodiments herein to extend the application of Partial Wave Spectroscopic microscopy to the study of temporal dynamics of the cellular nano-architecture. This technique allows for the rapid quantification of the nano-molecular organization in live eukaryotic cells without the use of exogenous labels. While live cell PWS alone is not molecularly specific, it is easily integrated with existing fluorescent methods, providing information that cannot be visualized by existing optical approaches. Furthermore, live cell PWS demonstrates that the nanoscale structure of chromatin evolves rapidly with time, which significantly transforms understanding in the field of the structure-function relationship between critical processes and chromatin structure, including DNA-repair, replication, and transcription. With this technique, experiments conducted during development of embodiments herein demonstrate that live cell DNA binding dyes, such as Hoechst 33342, cause rapid destruction of the higher-order chromatin structure at time-scales (seconds) not previously recognized. Paradoxically, this dye is ubiquitously used for the study of cell viability and the presence of DNA damage (ref. 40b). As a result, live cell PWS is a powerful tool for studying DNA damage/repair, chemotherapeutic efficacy in live cells, etc. Experiments were additionally conducted during development of embodiments herein to demonstrate the temporal dynamics of chromatin during continuous to UV light exposure, showing a transformation in both the temporal and physical properties of the chromatin nano-architecture during UV induced stress.

Additionally, experiments conducted during development of embodiments herein showed that live cell PWS allows for exploration of the factors affecting the chromatin nano-architecture by demonstrating differential responses in chromatin structure that depend on the mitochondrial membrane potential. In particular, this illustrates that mitochondrial function is intimately related to chromatin structure in real-time and that live cell PWS can for the first time act as a tool to further investigate the mechanisms of chromatin-metabolic interactions. Live cell PWS finds use, for example, as a supplement to super-resolution fluorescence techniques, providing quantifiable information about unstained cellular organization to examine the role of the nano-architecture on molecular interactions in live cells. PWS find use in exploring, for example: (1) the interaction between chromatin structure and mRNA transport; (2) the accessibility of euchromatin and heterochromatin to transcription factors(refs. 41b-43b; incorporated by reference in their entireties); (3) the relationship between chromatin looping, as measured by techniques such as Hi-C, to the physical chromatin structure (refs. 6b, 12b, 44b; incorporated by reference in their entireties); (4) why and how high-order chromatin structure changes in cancer (refs. 45b; incorporated by reference in its entirety); (5) the role of nuclear architecture as an epigenetic regulator of gene expression (6, 12, 44); (6) the effect of metabolism on chromatin structure (refs. 36b, 46b; incorporated by reference in their entireties); and (7) the role of chromatin dynamics in stem cell development (refs. 47b, 48b; incorporated by reference in their entireties); etc.

### Materials/Methods

### Live Cell Partial Wave Spectroscopic Imaging

Prior to imaging, media within petri dishes was exchanged with fresh, RPMI-1640 Media (lacking phenol red pH indicator, purchased from Life Technologies) supplemented with 10% FBS (Sigma Aldrich, St. Louis Mo.). For DNA fragmentation experiments, live cell PWS images were acquired at room temperature (22° C.) and in trace CO₂ (open air) conditions for cells subsequently stained with Hoechst 33342. During acquisition of time series data (UV and Controls, metabolic perturbation), cells were maintained at physiological conditions for the duration of the experiment. For imaging, a reference scattering spectra was obtained from an open surface of the substrate coverslip immersed in media prior to any cellular imaging to normalize the intensity of light scattered for each wavelength at each pixel. Σ is defined as the spectral standard deviation of our measured reflectance intensity normalized to this reference scattering spectra from the substrate-media interface. For Phase Contrast imaging, cells were grown and maintained in the same conditions as cells used for live cell PWS, but images were acquired with a 40× air objective and a transmission illumination beam. Likewise, for wide-field fluorescent imaging, cells were grown in the same conditions but pre-incubated with Hoechst 33342 for 15 minutes prior to imaging. To study the effects of UV illumination on cellular structure and function, cells were continuously exposed to UV light produced from an Xcite 120 LED light source (Excelitas, Waltham, Mass.) by removing the 500 nm long-pass filter from the illumination path (measurements were performed in triplicate, n=19). For Hoechst induced DNA damage experiments, significance was determined using Student's T-test with unpaired, unequal variance on nuclear Δ(Σ) between the conditions indicated in the experiment in both Mathematica v.10 and Microsoft Excel (Microsoft, Redmond, Wash.) with n=146 for Hoechst stained HeLa cells from 11 replicates and n=87 for Hoechst stained CHO cells from 5 replicates. For mitochondrial membrane depletion experiments, significance was determined using a two-tailed, paired Student's T-test on nuclear Σ before and after CCCP treatment using Microsoft Excel (Microsoft, Redmond, Wash.) with n=31 for HeLa cells from 6 independent experiments and n=159 for CHO cells from 5 independent experiments. Each experiment consists of 1-10 independent fields of view for analysis. Sequences of pseudo-colored live cell PWS images were merged into movies using ImageJ. All pseudo-colored live cell PWS images were produced using Matlab® v. 2015b using the Jet color scheme with the ranges indicated in the figure legend. All cells were purchased from ATCC (Manassas, Va.) unless otherwise noted and imaged in their cell appropriate media supplemented with 10% FBS. Human Umbilical Vein Endothelial Cells (HUVEC) were purchased from Lonza (Walkersville, Md.) and grown under cell appropriate media formulation on poly-1-lysine coated glass imaging dishes.

### Co-Localization

Fluorescence co-localization of organelle specific stains with live cell PWS imaging was performed through manual image alignment of mean-reflectance images produced by live cell PWS acquisition of unstained cells to the cells at the time of acquisition. Background intensity was removed using ImageJ® with using a rolling average of 50 pixels for nuclei and 75 pixels for mitochondria. Threshold intensities for the aligned fluorescence images were then calculated by FindThreshold function in Mathematica® version 10 utilizing Otsu's algorithm. Co-localized images were produced by the binary mapping of fluorescent images for each stain, pseudo-colored, and scaled by the live cell PWS Σ intensity.

### H2A.X Phosphorylation

Co-registration of live cell PWS imaging and DNA strand damage using phospho-histone H2A.X was performed by immunofluorescent staining on three independent experiments. Cells were fixed for 20 min with 4% paraformaldehyde at room temperature, washed twice with phosphate buffered saline and a permeabilization/blocking step was performed with 0.1% Triton X-100 in 1% bovine serum albumin (Sigma Aldrich, St. Louis Mo.) for 20 min. Cells were again washed twice with PBS and then incubated with AlexaFluor 488 conjugated to anti-γH2A.X (serine 139 residue) rabbit monoclonal antibody (Cell Signaling, Beverly, Calif.) for 30 minutes. Following incubation with the antibody, cells were imaged using the FITC-EGFP filter on the live cell PWS microscope.

### Mitochondrial Membrane Potential Perturbation

HeLa and CHO cells were grown and prepared for live cell imaging as previously described. Cell measurements for a single field of view were sequentially obtained for 3 minutes prior to treatment with CCCP. HeLa (n=31 from 6 independent experiments) and CHO (n=159 from 5 independent experiments) cells were treated with 10 μM for 15 minutes and imaged before and after treatment. Mock treated cells were incubated with 0.01% DMSO to account for the effect of DMSO solvent on the cells. No significant changes were observed in the mock treated cells for either cell line. Mitochondrial membrane potential ΔΨm was measured by flow cytometry (BD LSRII at the Northwestern Flow Cytometry Core) for tetramethylrhodamine (TMRE, purchased from Life Technologies, Carlsbad Calif.) stained cells. In brief, cells were trypsinized and immediately stained with 50 nM of TMRE for 30 minutes. Cells were washed twice with PBS after staining and suspended in 1 ml of PBS. CCCP treated cells were treated for 15 minutes to replicate conditions during live cell live cell PWS imaging. At least 20,000 cells were selected by forward and side scattering channels for each group, with a double elimination of doublets from the final analysis. Mean TMRE intensity from each replicate population was used for representative comparison between treated and untreated groups.

### Results

An exemplary live cell PWS instrument was built into a commercial inverted microscope equipped with a broadband illumination and a tunable spectral collection filter. With this configuration, the live cell instrument utilizes the glass-cell interface to produce the requisite interference signal that allows for the study of underlying nanoscale structure. In brief, the spatial fluctuations of refractive index (RI) produced by the macromolecular density distribution cause backscattering of incident light waves from the sample. Optical interference of the back-propagating light results in wavelength-dependent fluctuations in the acquired spectrally-resolved microscope image. The standard deviation of these spectra quantifies the internal structure of the sample with nanometer sensitivity (refs. 17b, 18b; incorporated by reference in their entireties). In cells, there are numerous variations in macromolecular density due to the spatial organization of macromolecules. Quantification of this nano-molecular density distribution is given by the statistical parameter, Σ, at each diffraction-limited pixel (refs. 17b, 18b; incorporated by reference in their entireties).

Σ, and the Disorder Strength (Ld which is Σ normalized by sample thickness), are proportional to two crucial characteristics of molecular organization at deeply subdiffractional length-scales: the characteristic length scale of macromolecular organization (Lc), and the standard deviation (6n) of the density (refs. 17b, 18b; incorporated by reference in their entireties). In a fractal media, such as chromatin, the characteristic length scale of macromolecules can be alternatively evaluated through the fractal dimension, D, which is proportional to Σ. Thus, Σ measured from chromatin senses nanoscale changes in its fractal organization. Previous molecular dynamics simulations have further confirmed that increases in δn*Lc correspond to an increase in macromolecular compaction, and experimental results have shown that this increase within the nucleus quantitatively describes an increase in chromatin heterogeneity (refs. 20b-22b; incorporated by reference in their entireties).

As a representation of the nanoscopic topology detected by live cell PWS, 10 nm “beads on a string” chromatin fibers were used as a model (FIGS. 5A&B) (ref. 22b; incorporated by reference in its entirety). In this model, changes in the nanoscopic structure of higher-order chromatin that have the same nanoscopic average mass density but have starkly different nanoscale organization are considered: differentially compacted (FIG. 5a) and diffusely compacted (FIG. 5B) DNA fibers. In both cases, images produced from conventional light microscopy techniques cannot capture information about the nanoscale topology these differential states (FIGS. 5C&D). PWS provides information about their sub-diffractional organization. To demonstrate sensitivity to these structures, Σ is computed directly, accounting for the physical properties of the live cell system (refs. 17, 18; incorporated by reference in their entireties). As is shown in FIG. 1E, differentially compacted chromatin (FIG. 5A) produces a much higher Σ than diffusely compacted chromatin (FIG. 5F). Consequently, regions that result in high Σ in live cells would be the heterogeneous, differentially compacted regions likely resulting from the formation of local heterochromatin domains neighboring decompacted euchromatin (FIG. 5A). Conversely, homogeneous regions of chromatin would result in low Σ.

While this instrument configuration was optimized to allow live cell imaging with multi-modal acquisition, including wide-field fluorescence and phase contrast microscopy, it has an appreciably weaker reference-interference signal than that produced in traditional PWS cytology and a much higher objective collection numerical aperture. Therefore, the nanoscale sensitivity of live-cell PWS was validated by using rigorous Finite-Difference Time-Domain computations to numerically solve Maxwell's equations without approximations simulating the nanoscale-complex spatial distribution of molecular density in live cells. These computations were employed to study the effect of the RI mismatch using sapphire as a high-RI substrate on the interference signal, and to compare the effect of numerical aperture on Σ. The FDTD simulations allowed for optimization of the configuration of signal acquisition in order to provide nanoscale sensitivity to intracellular structure at length scale between 20 and 200 nm.

Without the use of exogenous labels, this technology achieves high-contrast images using Σ that delineate nuclei from cytoplasm due to the intrinsic differences in their nano-architecture (FIG. 5G). Due to its multi-modal design, in some embodiments, exogenous and endogenous labels are used to co-localize specific molecular markers or organelles (FIG. 5H). Live cell PWS acquisition yields a three-dimensional data cube, I(λ,x,y), where λ is the wavelength and (x,y) correspond to pixel positions across a 10,000 μm² field of view, allowing multiple cells to be imaged simultaneously. Acquisition of the full cell-reference interference spectrum (500-700 nm) for spectral analysis takes under 30 s, with each wavelength collection produced from <100 ms exposures. Using a reduced wavelength approach to sub-sample the interference spectrum, this can be further reduced to under 2 s per acquisition (ref. 23b; incorporated by reference in its entirety). Even with full spectral collection (FIGS. 5J&K; incorporated by reference in its entirety), live cell PWS provides rapid, quantitative visualization of cellular structures within a single field of view for dozens of cells simultaneously for multiple cell lines (FIG. 5J, 20 HeLa cells captured in ˜30 seconds; FIG. 5K, 36 MES-SA cells captured in ˜15 seconds). Indeed, a feature of this rapid acquisition is the capacity to directly study the underlying heterogeneity of both chromatin structure and its temporal evolution within the cell population over time. Likewise, as a label-free technique using low illumination intensity, live cell PWS allows for the study of various time evolving processes on the structure of cells in general, and chromatin in particular for different cell types over extended periods of time.

Live cell PWS has a broad utility as a tool for studying the complex relationships between cell function and chromatin nano-organization. Using live cell PWS, experiments conducted during development of embodiments herein demonstrate that the addition of Hoechst 33342 to living mammalian cells transforms the nano-organization of chromatin at the time scale required for imaging and that subsequent excitation induces fragmentation of the chromatin nano-architecture within seconds. This is apparent, as an overall decrease in the Σ was observed after irradiation, indicating homogenization and decompaction of chromatin across the entire nucleus (FIG. 6A). Additionally, these effects persist for longer durations, lasting at least 15 minutes indicating that the once fragmented, chromatin in the presence of the dye does not immediately reassemble suggesting these changes could be irreversible. To control for the effects of ionizing UV radiation required for Hoechst excitation, a mock-staining (M-S) experiment was performed in which the nuclear changes in cells incubated with Hoechst 33342 were compared to those exposed to UV light alone. In the M-S cells, there was not an observable change in cellular or chromatin structure during the short illumination time required for Hoechst excitation, indicating preservation of the original chromatin structure (FIGS. 6B&C). Quantitatively, M-S cells showed no significant change in mean-nuclear Σ after a few seconds of UV exposure, whereas the Hoechst-stained cells display a 17.01% decrease in HeLa (99% confidence interval Hoechst (−18.5%, −15.6%), p-value<0.001) between mock and Hoechst stained cells with n=146 cells from 11 independent experiments for Hoechst stained cells and n=68 cells from 6 independent experiments for M-S cells (FIGS. 6B&C). In Hoechst-stained cells, all nuclei demonstrate a negative change in the mean nuclear Σ after UV exposure, whereas the M-S cells display a narrow, zero centered distribution after UV exposure (FIG. 6E). In both M-S and Hoechst-stained cells, cytoplasmic Σ did not change following UV exposure (p-value>0.05). Similar results were observed for Chinese Ovarian Hamster (CHO) cells with M-S cells displaying no change, whereas Hoechst stain cells experience a −7.1% decrease (99% confidence interval Hoechst (−9%, −5%), p-value<0.001 between M-S and Hoechst stained cells, n=127 cells for M-S, n=87 for Hoechst-stained from 5 independent experiments each), demonstrating this effect occurs independent of the cell type.

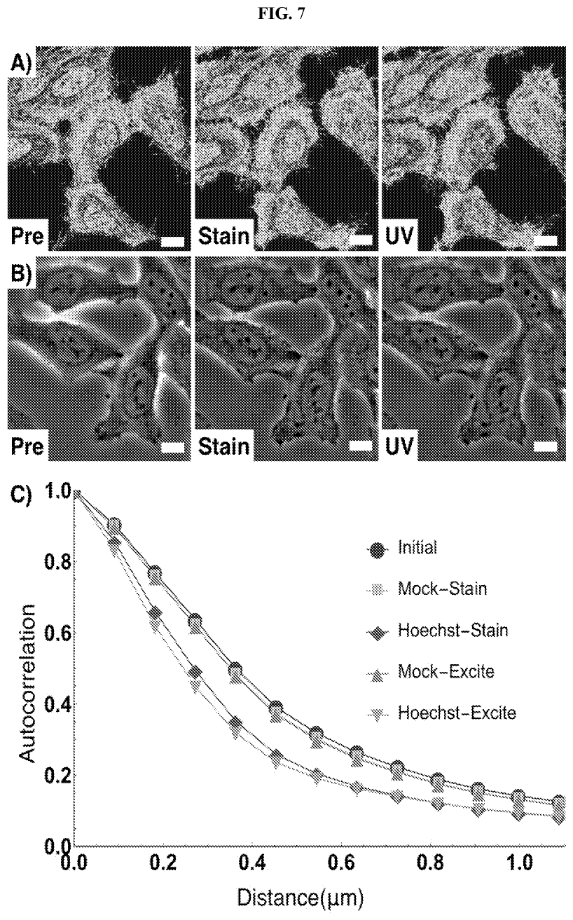

Experiments conducted during development of embodiments herein to test whether the decrease in the mean nuclear Σ was due to the homogenization of the higher-order chromatin organization caused by DNA fragmentation and the resulting nuclear remodeling. Experiments utilized a γ-H2A.X-Alexa488-conjugated antibody to independently monitor the fragmentation of DNA. In Hoechst-stained cells, a drastic accumulation of the γ-H2A.X antibody was observed, whereas little or no localization was observed in the M-S control nuclei (FIG. 6D). Additionally, transmission electron microscopy on Hoechst-stained and M-S cells exposed to UV light showed an increase in micron-scale dense chromatin clumps compared with untreated cells (FIGS. 6F&G). This further demonstrated that immediate DNA fragmentation was induced by Hoechst-33342 excitation, a phenomenon that is detectable by live cell PWS in real-time without the need for exogenous labels. Subsequent experiments compared live cell PWS with phase contrast microscopy to determine if live cell PWS provides information not detectable by other standard, label-free optical modalities (FIGS. 7A&B). With phase contrast microscopy, no changes in the cell or nuclear structure were detected after excitation of Hoechst 33342 due to its diffraction-limited resolution (FIG. 7B). While electron microscopy cannot be performed on live cells, these experiments demonstrate that photo-excitable molecules disrupt the chromatin nano-architecture, which is uniquely detectable in real-time in live cells by live cell PWS.

Experiments were conducted during development of embodiments herein to investigate the effects of Hoechst staining on the spatial transformation of chromatin nano-organization as measured by live cell PWS. In particular, the spatial distribution of Σ across the nucleus was analyzed by calculating the two-dimensional spatial autocorrelation, which measures the change in the pixel-to-pixel variability as a function of distance. An increase in the spatial autocorrelation indicates that the nanoscale structure at one pixel has become similar to its neighboring pixels, while a decrease indicates a more locally heterogeneous structure. The size of these clusters of similar nanoscale structures was significantly decreased between 100 nm and 1 μm after both the addition of Hoechst and its excitation (n=40 from 3 independent experiments) (FIG. 7C). This indicates an increase in the spatial microscopic heterogeneity of nanoscopic heterogeneity of the nuclear nanoscale structure (Σ) after Hoechst addition and excitation. Consequently, it was found that Hoechst causes a global alteration in the chromatin nano-architecture independent of its excitation. Not only does this study demonstrate the ability of live cell PWS to sense the heretofore undetectable in live cells alterations in chromatin structure such as double strand DNA breaks, but it also illustrates some of the limitations of the extrinsic labeling approaches, such as Hoechst: even though they have traditionally been used for live cell imaging, these labels alter chromatin structure and lead to DNA damage, which in turn may leads to a perturbation of cell function.

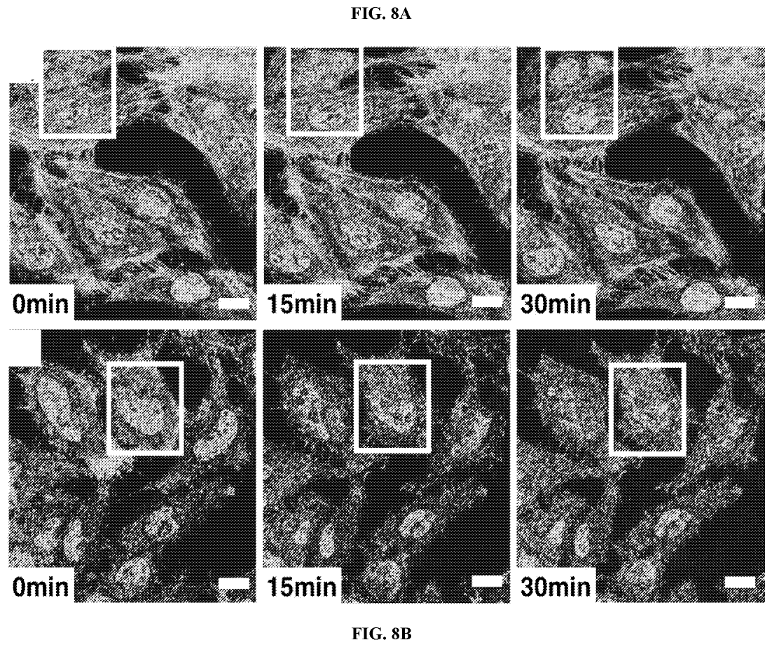

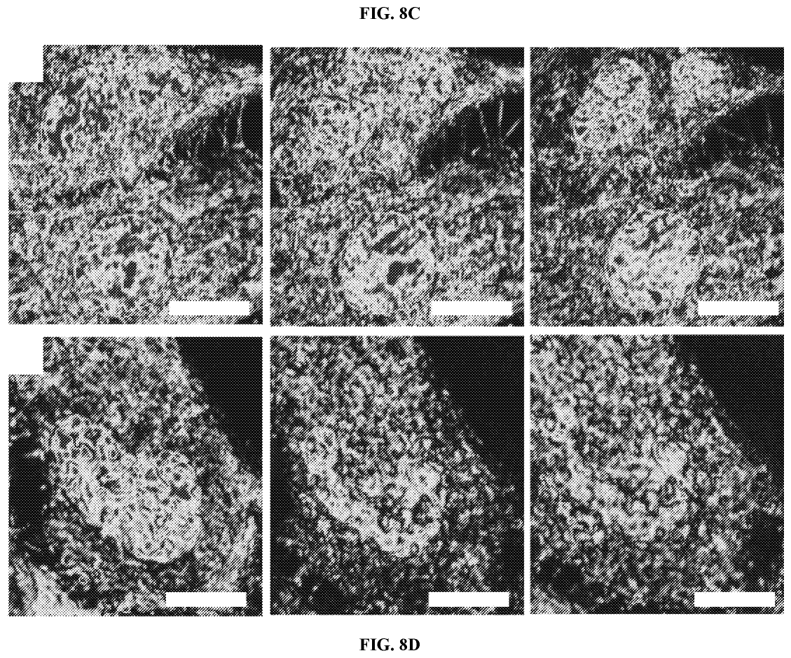

Live cell PWS was next applied to study the temporal dynamics of the cellular nano-architecture under normal growth conditions (FIG. 8A) in comparison to cells exposed to continuous UV light (FIG. 8B). UV light is known to cause DNA damage, generate reactive oxygen species, alter receptor-kinase function, and disrupt the cellular membrane. Under normal conditions, chromatin structure can evolve rapidly, with whole-scale changes occurring in minutes (FIG. 8C). While the nanoscale topology of chromatin rapidly evolves within any given cell, the organization across the population overall remains stable under normal conditions. In comparison, during continuous UV exposure over 30 minutes, higher-order chromatin structure is degraded after few minutes of exposure (FIG. 8D), with pronounced variations in structure over time from cell to cell. There are numerous phenomena that occur to the cellular nano-architecture during continuous UV exposure across a distribution of time scales.

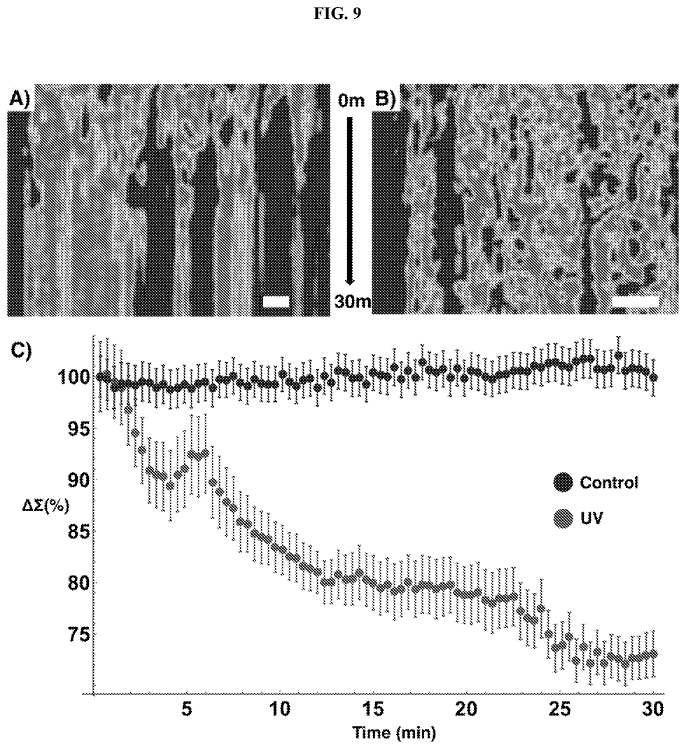

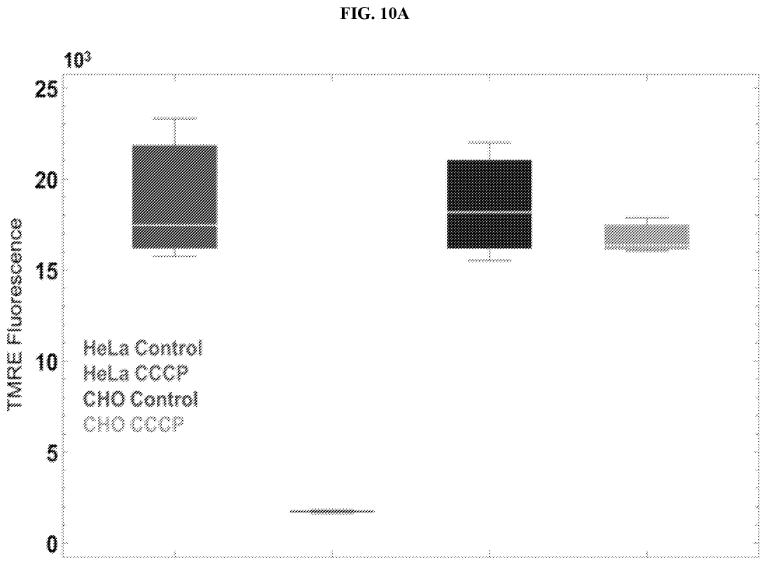

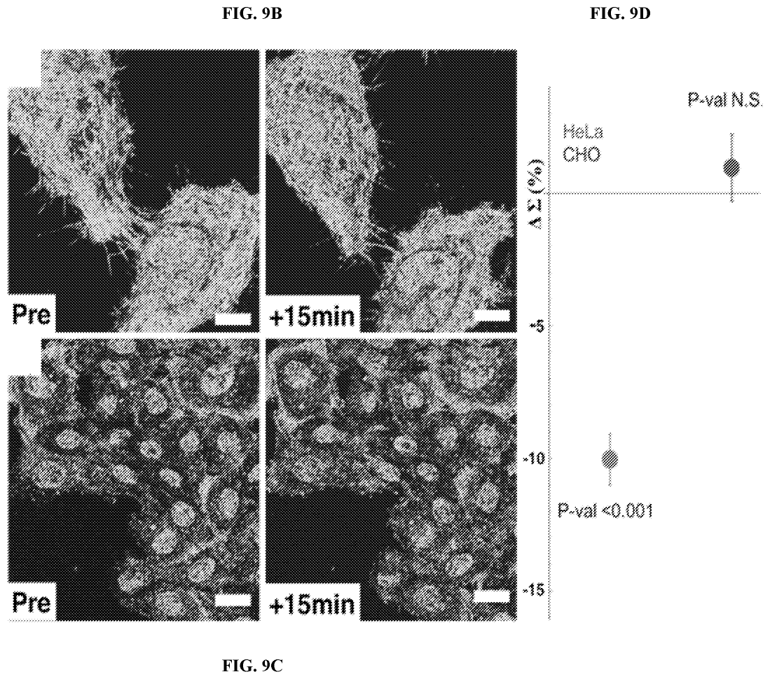

Over the course of 2-3 minutes, there are minimal changes in chromatin and cellular topology due to UV light exposure. However, after approximately ˜3 minutes, the chromatin of some cells exposed to UV light undergoes rapid, directional increase in heterogeneity that corresponds with the formation of micron scale homogeneous domains (FIG. 8E). Concurrently, the cytoplasm of the cell is transformed, with cell-cell adhesions retracting and a retreating waveform spreading from the cell periphery toward the nucleus. Finally, a near-instantaneous transition occurs within the cytoplasm, with the changes in the cytoplasmic and chromatin nanostructure spontaneously arresting 20 minutes after exposure (FIG. 8E). To capture these temporal dynamics in nanostructure, a kymograph analysis was performed using ImageJ® of a representative cell exposed to UV light in comparison to a control cell. As is shown in FIG. 9A, over the 30 minutes of exposure to UV, micron-scale homogeneous domains form within the nucleus and the temporal evolution of nanostructure ceases. In comparison, control cells display continuous transformation, with homogeneous and heterogeneous domains transiently forming and dissipating over the time frame of a few minutes (FIG. 9B). The formation of these large, homogeneous domains that lack high-order structure dominate, resulting in an overall decrease in Σ (average decrease at 30 min of 26.9% calculated from 19 nuclei from 3 independent experiments) (FIG. 9C). Even under control conditions, some cells rapidly demonstrate global changes in their chromatin topology. Despite these rapid alterations, the overall chromatin structure of the population displays minimal changes over the course of 30 minutes (average 0.2% decrease Σ in from 32 nuclei from 2 independent experiments) (FIG. 9C).

Experiments were conducted during development of embodiments herein to demonstrate the broad utility of live cell PWS as a tool for studying the complex relationships between cell function and chromatin nano-organization by observing the effect of alteration of cellular metabolism on higher-order chromatin architecture. Studies have shown that the cellular metabolic activity is intimately linked to cell replication, tumor formation, DNA damage response, and transcriptional activity (refs. 34b-37b; incorporated by reference in their entireties). Recent fluorescence microscopy studies have suggested that impairment of cellular metabolism induces rapid (<15 min) transformation of chromatin (refs. 38b, 39b; incorporated by reference in their entireties). However, these studies often require the production of specialized transfection models (H2B-GFP) or the use of DNA-binding dyes such as Hoechst 33342, and as such, are limited in their ability to study multiple cell lines and/or over significant periods of time without perturbing the natural cell behavior (refs. 38b, 39b; incorporated by reference in their entireties).

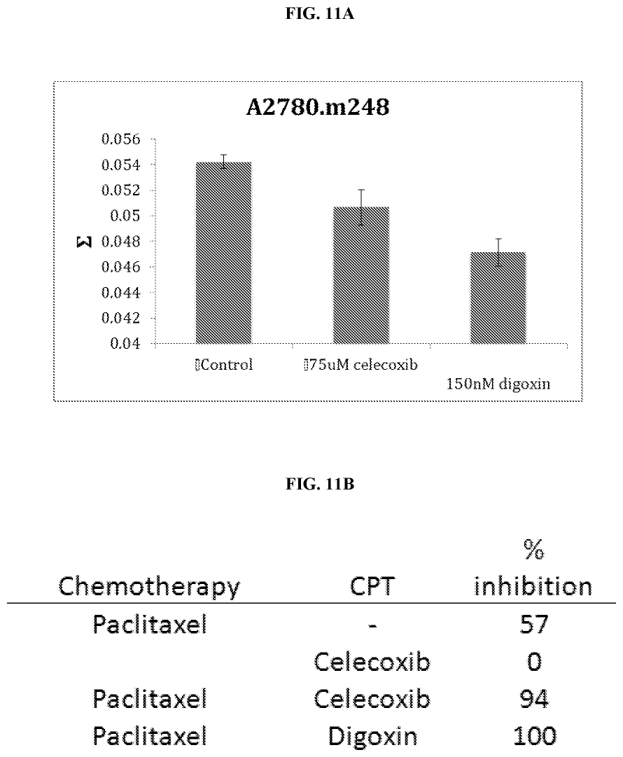

In order to study the link between chromatin structure and mitochondrial function, the protonophore, Carbonyl cyanide m-chlrophenyl hydrazine (CCCP), was employed, which is widely used for studies of mitochondrial function due to its disruption of mitochondrial membrane potential (ΔΨm). To explore the role of ΔΨm reduction on the immediate transformation of the chromatin nano-architecture in live cells, two cell lines, HeLa and CHO, were used. Following addition of 10 μM CCCP, HeLa cells rapidly lost ΔΨm whereas CHO cells displayed no significant change as gauged by TMRE fluorescence (FIG. 10A). After 15 minutes of treatment with CCCP, it was found that addition of 10 μM CCCP produced rapid transformation of chromatin structure in HeLa cells but not in CHO cells (FIG. 10B). In HeLa cells, a decrease in nuclear Σ was observed, indicating homogenization and decompaction in the chromatin structure. Conversely, in CHO cells, no statistical change was observed in chromatin compaction and heterogeneity (FIG. 10C). Quantitatively, HeLa nuclei showed a 10% decrease in mean-nuclear Σ after CCCP (p-value<0.001, n=31 from 6 independent experiments), whereas the CHO cells displayed no significant increase in mean-nuclear Σ (n=159 cells from 5 independent experiments) (FIG. 10D). This transformation indicates that the depletion of mitochondrial membrane potential induces rapid decompaction and homogenization of chromatin nanostructure. Disruption of the ΔΨm has numerous effects, including the inhibition of mitochondrial ATP synthesis, changes in the production of reactive oxygen species (ROS), altered signal transduction, as well as modification of other mitochondrially produced metabolites (e.g., acetyl and methyl transfer groups). These results demonstrate that the change in Ψm rapidly regulates the nanoscale organization of chromatin, possibly resulting in the observed decreased proliferative potential of cells over time.

### Example 2

Test compounds were screened for CPT activity using live cell PWS imaging. Control and 30-60 minute CPT-treated cells were imaged with live cell PWS to determine their effect on nuclear nanoscale heterogeneity (See FIG. 11A for exemplary tested compounds). A significant decrease in chromatin heterogeneity, as measured by a decrease in mean nuclear sigma by live cell PWS, demonstrates CPT activity for the compound. Compounds that led to this decrease in chromatin heterogeneity were then tested with traditional chemotherapeutic agents.

Cell survival, as measured by surface coverage, was used to determine chemotherapeutic efficacy of cells treated with CPTs and chemotherapeutic agents (See FIG. 11B for exemplary tested compounds). Multiple petri dishes were plated on the same day at the same density and treated after the cells were adhered. For each tested CPT, there were separate dishes for control cells, cells treated with CPT alone, with chemotherapy alone, and with CPT and chemotherapy in combination. After 48 hours, bright field transmission was used to determine surface coverage for each condition. For each dish, 3×5 fields of view (205×205 uM each) were captured and % coverage was calculated for each field of view. The average of all the fields of view was then calculated for an overall representative percent surface coverage. The difference in % coverage was then calculated for each treated condition relative to the control.

For example, in the ovarian cancer cell line A2780.m248, live cell measurements celecoxib and digoxin showed a decrease in mean nuclear sigma for both celecoxib- and digoxin-treated cells (FIG. 11B). These compounds were tested in combination with the chemotherapy drug paclitaxel. Paclitaxel alone only resulted in 43% cell death (as measured by surface coverage normalized to the control cells). However, paclitaxel in combination with celecoxib led to 94% inhibition and paclitaxel in combination with digoxin led to 100% inhibition.

### Example 3

### Optical Imaging to Evaluate CPT Candidates

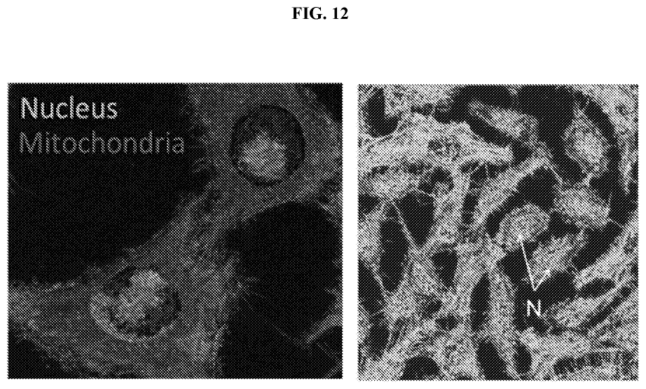

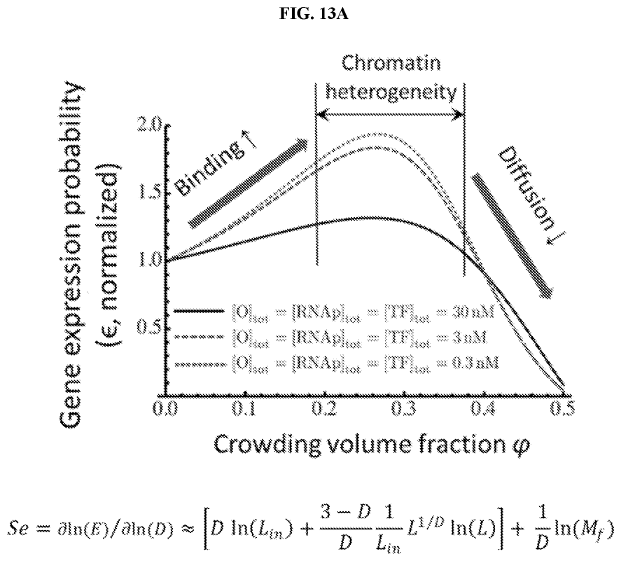

Supra-nucleosomal chromatin folding at length scales from about 10 nm (nucleosomes) to ˜200 nm (the size of the chromatin fractal globule) is a key determinant of transcriptional heterogeneity. Assessment of agents capable of modulating this level of chromatin folding requires the ability to image macromolecular organization at the nanoscale. The resolution of optical microscopy is limited by diffraction to >200 nm. Recently, a major breakthrough has been made in the emergence of super-resolution fluorescence microscopy. However, these techniques require the use of exogenous labels, and exogenous fluorophores tend to perturb cell function, and these techniques cannot be used to study the entire chromatin nanoscale environment as only specific molecular species can be labeled at any given time (Ref. 19c; incorporated by reference in its entirety). Partial wave spectroscopic (PWS) microscopy provides a nanoscale imaging technique for live cell imaging (FIG. 12) (Refs. 20c-21c; incorporated by reference in their entireties). The main principle of PWS is a previously overlooked phenomenon that while sub-diffractional structures are not resolvable, they are still detectable through the analysis of interference of elastically scattered light. PWS measures the statistics of macromolecular density distribution within live or fixed cells with sensitivity to 20-200 nm length scales, which is ideally suited for the characterization of supra-nucleosomal chromatin topology (Ref. 21c; incorporated by reference in its entirety).

### Chromatin Heterogeneity is a Critical Event in Carcinogenesis

The three-dimensional (3D) organization of the genome is a subject of active research, and a number of models have been proposed. Technologies such as Hi-C led to the development of the crumpled fractal globule model, which was proposed to replace earlier constructs such as the random polymer model (Refs. 22c-23c; incorporated by reference in their entireties). More recently, alternative models have emerged including the loop extrusion and the random loop models. Regardless of the exact configuration and the kinetics of chromatin folding, any 3D chromatin arrangement can be described statistically by its auto-correlation scaling. Experimental evidence indicates that the higher order chromatin organization can be characterized as a power-law scaling (fractal) media with a fractal dimension, D<3 (Refs. 24c-25c; incorporated by reference in their entireties), including ex vivo sensing techniques such as neutron scattering and chromosome conformation capture (3C, 5C, Hi-C) (Refs. 22c, 23c, 26c; incorporated by reference in their entireties) and in vitro imaging such as transmission electron microscopy (TEM) (Ref. 27c; incorporated by reference in its entirety), PWS (Ref 28c; incorporated by reference in its entirety), fluorescence correlation spectroscopy (Ref 25c; incorporated by reference in its entirety), and photon localization microscopy (PLM) (Refs. 25c, 27c-31c; incorporated by reference in their entireties). A fractal media is not synonymous with a fractal globule (ref. 32c; incorporated by reference in its entirety), but instead refers to the scaling of the auto-correlation function of the chromatin arrangement (Ref. 30c, 33c; incorporated by reference in their entireties); loop extrusion (Ref. 34c; incorporated by reference in its entirety) and random loop models (Refs. 35c-36c; incorporated by reference in their entireties) are likewise fractal media because they have a self-similar scaling (ref. 37c; incorporated by reference in its entirety).

Chromatin Folding and GGL (Refs. 11c-12c; Incorporated by Reference in its Entirety)

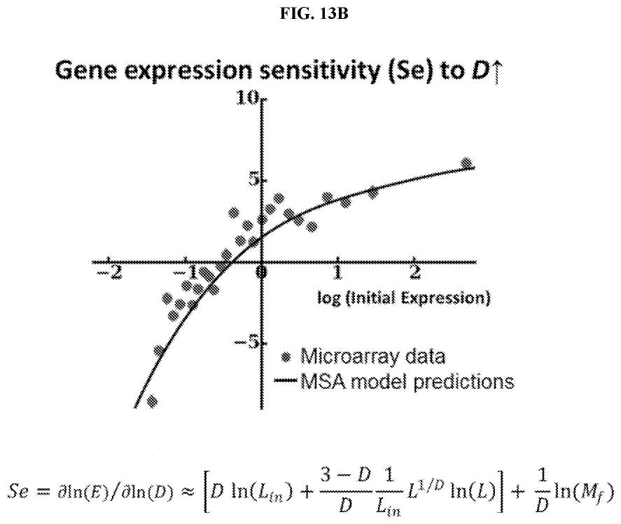

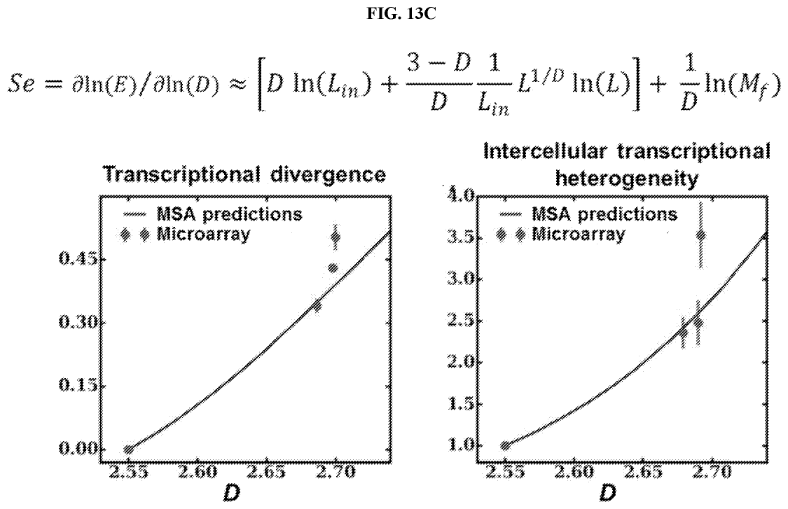

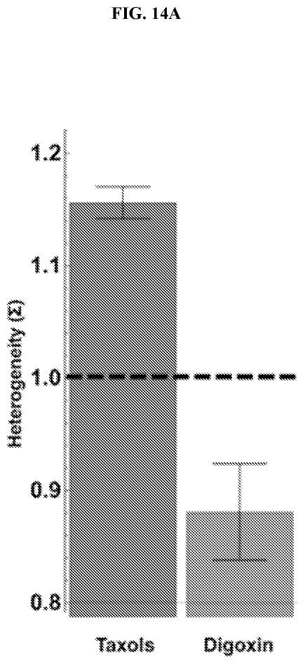

Essentially every molecular event involved in transcription (e.g., surface area of chromatin interface, diffusion rates and the binding constants of transcription factors, etc.) are modulated by the local density of chromatin (Ref 1c; incorporated by reference in its entirety). Gene expression is a probabilistic event and the rates of nearly every molecular process involved in transcription can be modulated by orders of magnitude by changing the nanoenvironment within the transcriptional interaction volume (Ref 1c; incorporated by reference in its entirety). Physical mechanisms determine the profound regulatory role of chromatin folding on gene transcription. A shift of chromatin to a high-heterogeneity state (D↑, Σ↑) leads to a greater dynamic range of transcriptional states (“transcriptional divergence”) and intercellular transcriptional heterogeneity (FIG. 13) (Ref 12c; incorporated by reference in its entirety)

Multi-Systems Analysis (MSA), which incorporates Brownian Dynamic (BD), Molecular Dynamics (MD), and Monte Carlo (MC) simulations integrated with mathematical modeling of gene expression predicts that D↑ leads to an increase in the accessible surface area of chromatin, which amplifies global gene expression (Refs. 1c, 11c-12c; incorporated by reference in their entireties). Concomitantly, D↑ also increases the standard deviation of local crowding (Ref 12c; incorporated by reference in its entirety), which differentially suppresses gene transcription with an impact that is inversely dependent on a gene's initial transcription rate. This is because the rate of transcription per accessible surface area, ε, is a non-monotonic function of the molecular crowding (φ) within the transcription interaction volume due to the competition of the two effects of crowding: increased molecular binding rates—this facilitates transcription through the stabilization of transcription complexes—and decreased molecular diffusion, which lowers the probability of formation of the complexes (FIG. 13a). D↑ increases the standard deviation of local crowding, which in turn decreases ε as now genes are exposed to a wider range of crowding conditions for which ε is not at its maximum (FIG. 13a). The non-monotonic relationship ε(φ) is modulated by the probability of a gene being transcribed: highly expressed genes already have optimized binding due to gene-specific factors such as histone interactions and their ε is not reduced significantly, whereas ε for initially suppressed genes is more affected (FIG. 13b). The net effect is the simultaneous activation of already highly-expressed genes and the suppression of partially-suppressed genes and, thus, a greater range of transcriptional products; that is, transcriptional divergence.

Due to the exposure of any given gene to varied nanoenvironments across a cell population, D↑ facilitates intercellular transcriptional heterogeneity.

Due to the non-linear interactions among genes within a gene network, a rise in D makes gene networks increasingly heterogeneous (gene network heterogeneity).

MSA modeling (Ref. 1c; incorporated by reference in its entirety) was able to accurately predict these effects (Refs. 11c-12c; incorporated by reference in its entirety). FIG. 13 shows MSA prediction of the gene expression changes in response to a change in D compared against microarray data. D was measured by live-cell PWS (Ref. 15; incorporated by reference in its entirety) within 30 min after application of transcription-perturbing treatments (differential FSB, EGF, and PMA, as well as SWI/SNF chromatin remodeling inhibition through sh-ARID1a). Although these treatments act through very different mechanisms, the resulting pattern of gene expression is primarily determined by D as the data for all treatments follow the same curve. (The model is not a fit to the microarray data: all model parameters were taken from prior literature (Refs. 3c, 38c; incorporated by reference in their entireties) This shows the critical role chromatin topology plays in the regulation of global patterns of transcription.

The heterogenization of chromatin nanoenvironment (D↑) allows cells to explore a greater transcriptional landscape (Refs. 10c, 39c; incorporated by reference in its entirety). A normal chromatin nanoenvironment restricts cells to a niche within the “space” formed by the ˜20,000 human genes, whereas the abnormal chromatin structure in cancer cells facilitates transcriptional exploration (Ref 10c; incorporated by reference in its entirety). This allows cancer cells to develop new traits, including chemoresistance, through the “discovery” of molecular pathways of drug resistance. Chromatin heterogenization does not compel cells to change their genome in any specific way; it simply modulates the barrier for functional changes to occur. Throughout tumor progression cancer cells must keep developing new traits, be it the induction of angiogenesis or finding strategies to evade the immune system, and chromatin heterogenization facilitates this process.

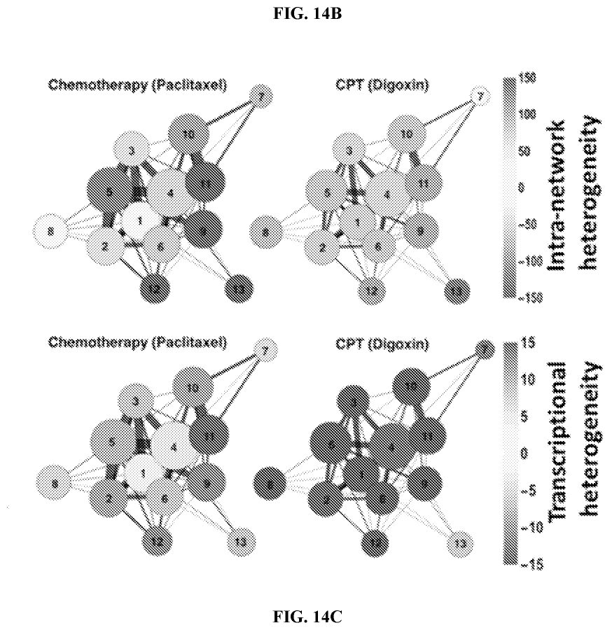

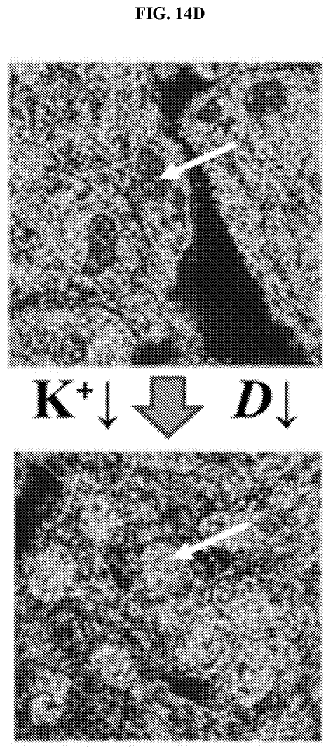

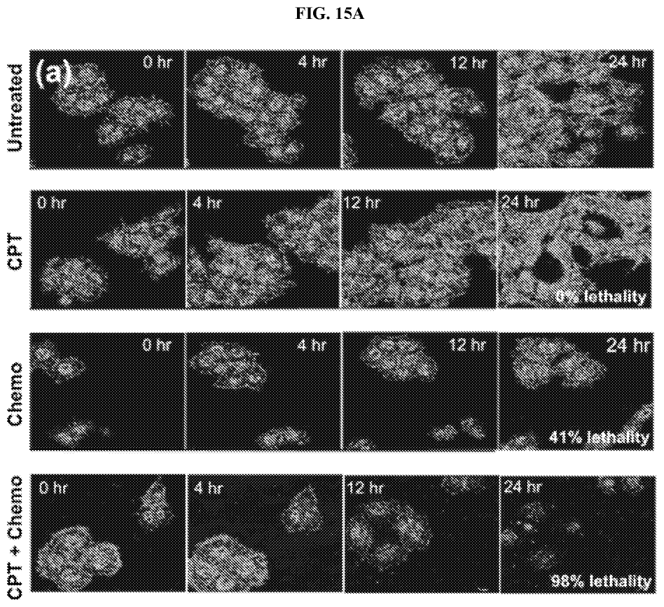

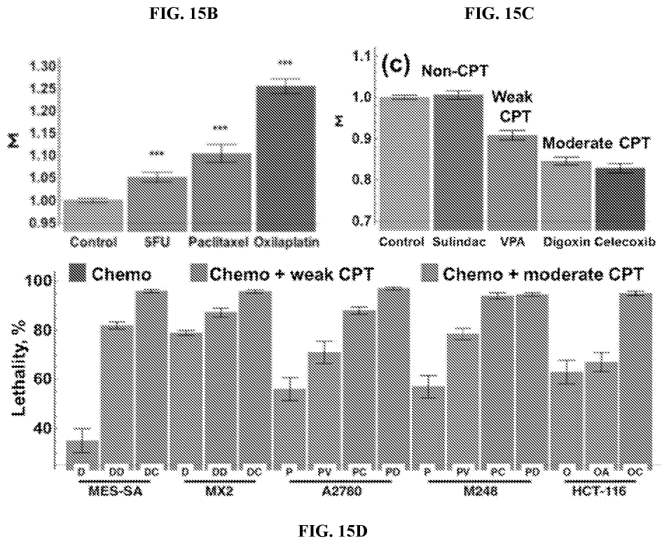

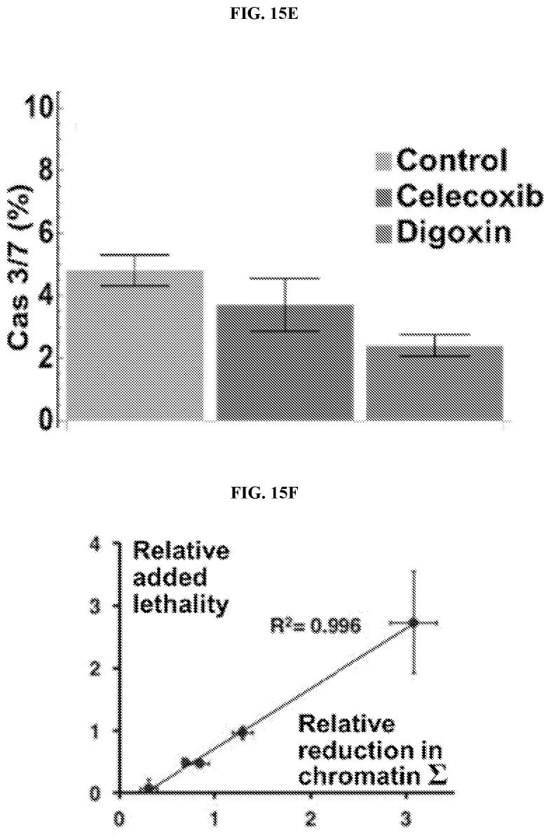

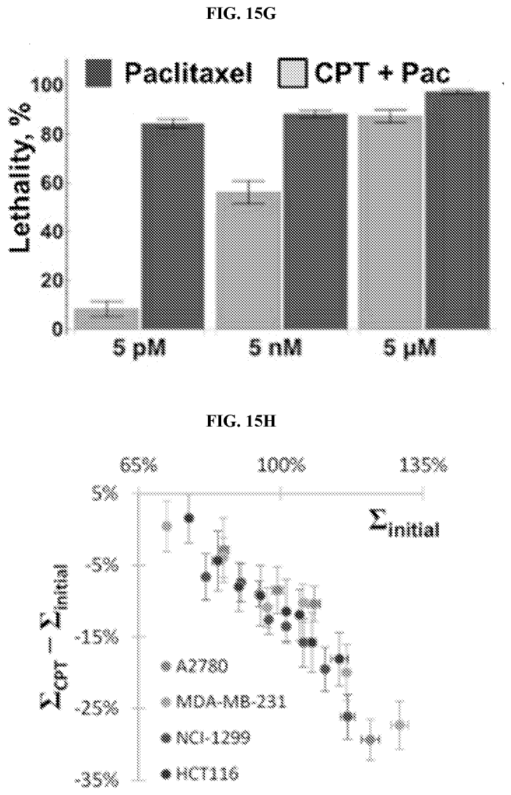

The application of chemotherapy itself increases chromatin D (measured by PWS) in every cell line studied to date (FIG. 15b). Single-cell sequencing has confirmed the predicted increase in transcriptional divergence, intra-network heterogeneity, and inter-cellular transcriptional heterogeneity in cancer cells within a few hours after exposure to chemotherapy compounds (FIGS. 14 & 15b). There are a multitude of transcriptionally-derived chemoresistance mechanisms including reduced drug accumulation and/or increased export, alterations in drug targets and signaling transduction molecules, repair of drug-induced DNA damage, and evasion of apoptosis (Ref. 2c; incorporated by reference in its entirety). New gene mutations are not always necessary for the development of drug resistance; a change in the expression of existing genes (e.g. MDR overexpression, suppression of apoptotic pathways) suffices. Transcriptionally diverse cells have an advantage in exploring new genomic states leading to the “discovery” of these pathways at time scales below the rate of cell division (Refs, 7c, 11c, 12c; incorporated by reference in their entireties).

### Regulation of Transcriptional Heterogeneity Through Chromatin Nanoenvironment

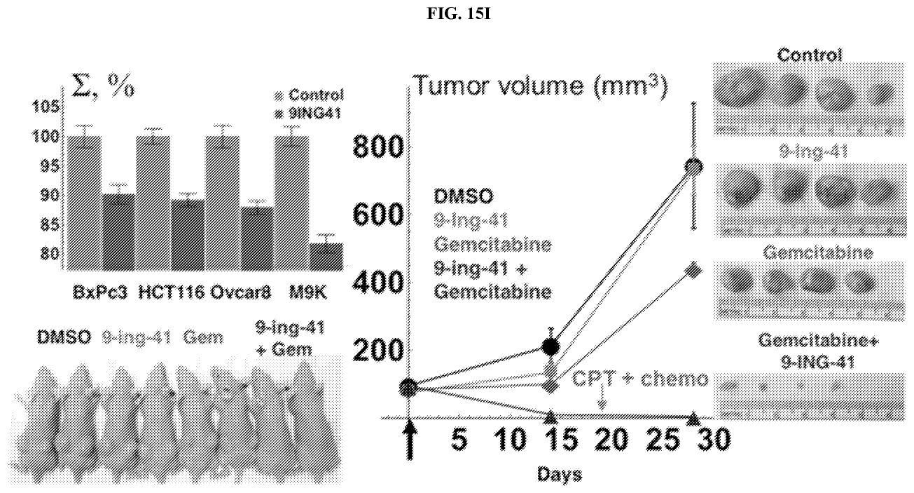

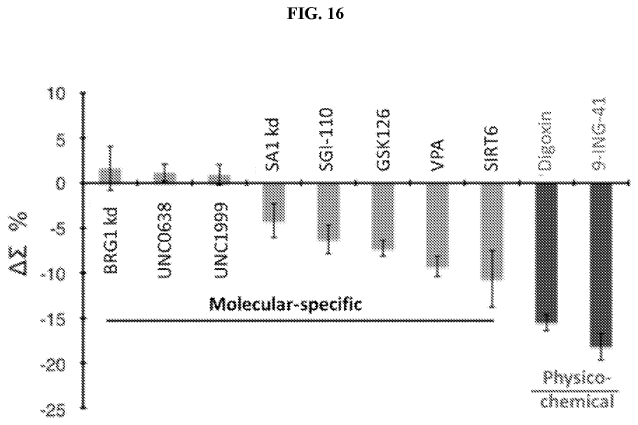

Experiments conducted during development of embodiments herein to demonstrate that the normalization of chromatin folding (D↓) decreases transcriptional heterogeneity. Although partial regulation of folding is achieved through molecular-specific chromatin remodelers (Refs. 12c, 40c; incorporated by reference in their entireties) (CTCF, SWI/SNF, histone modifications), a more efficient global regulation is governed by physico-chemical mechanisms (FIG. 16). A cardiovascular drug digoxin provides one such example. Administration of digoxin leads to D↓ (observed by PWS in cell culture within 30 min after application; this early time point was chosen to prevent confounding from new protein synthesis) and lower intracellular transcriptional divergence and intercellular heterogeneity, which was confirmed by single-cell sequencing (FIG. 14).

### Reduction of Chemoresistance Through Regulation of Chromatin Nanoenvironment

Experiments conducted during development of embodiments herein to demonstrate that CPT agents decrease cancer cells' ability to develop resistance in 12 different cell line models of colon (HCT-116), ovarian (A2780, A2780.M273, A2780.M248, OvCar8), leiomyosarcoma (MES-SA, MES-SA.MX2), breast (MDA-MB-231), pancreatic (AsPC-1, L3.6PL), mesothelioma (M9K), and lung (NCI-H1299) cancers. The cell lines were treated with standard (IC₅₀) doses of commonly used chemotherapy drugs (5-FU, Paclitaxel, Oxaliplatin, Docetaxel, Gemcitabine, FOLFIRI). In addition to the ionophores (cardiac glycosides digoxin, valinomycin), a number of other CPT agents have been identified based on their capacity to reduce chromatin folding, which was assessed by PWS (D↓, Σ↓) on live cells: several NSAIDs (celecoxib, aspirin), other compounds (e.g. EGCG, Curcumin), and a GSK3b inhibitor (9-ING-41) (FIGS. 15a, 15d & 15i). Chemotherapy alone was only partially effective, showing lethality rates from 6 to 54% (1—viable cell count after treatment relative to that for untreated cells) (FIGS. 15a, 15d, & 15g). On their own, CPTs did not induce apoptosis (FIG. 15e); however, combined CPT+chemotherapy treatment resulted in nearly 100% cancer cell death in all of the cancer cell lines with at least one CPT agent (FIG. 15d). Nearly 90% of cancer cells were eliminated with 0.1% of the IC₅₀ dose of chemotherapy when used in combination with a CPT (FIG. 4g). When chemotherapy was used alone, this level of cell death was obtained only at 1,000-fold of the standard dose (FIG. 15g). From this perspective, a non-toxic CPT is “equivalent” to a 10⁶-fold higher dose of chemotherapy. The efficacy of cancer cell lethality imparted by CPT agents was highly correlated with the ability of these compounds to reduce chromatin folding heterogeneity (r²=0.996), further confirming the role of chromatin folding as the common denominator of chemoresistance (FIG. 15f). The CPT effect on chromatin was proportional to the initial severity of heterogeneity (r²=0.962) (FIG. 15h), indicating that CPT is specific to cancer cells, without affecting normal cell populations. Experiments were conducted during development of embodiments herein to test CPT in vivo on patient derived xenographs (PDX) of pancreatic ductal carcinoma (FIG. 15i). GSK-3 inhibitor 9-ING-41 was utilized due to the fact that 9-ING-41 was a strong CPT across multiple cancer lines (colonic, pancreatic, ovarian, and mesothelioma). When treated with vehicle (DMSO), 9-ING-41 alone, or the standard of care chemotherapy agent, gemcitabine, the tumors continued to expand in size. However, the chemotherapy+9-ING-41 co-treatment caused a rapid decrease in tumor volume, leading to a remission of pancreatic tumors after 28 days (shrinkage in tumor volume to under 4% of its initial size).

Cytotoxic intervention results in increased variations in both chromatin folding and transcriptional heterogeneity; both are reversed by CPT agents. The robustness of the CPT adjuvant approach is highlighted by its capacity to potentiate cell elimination across a wide range of chemotherapeutic agents. CPTs synergize with microtubule depolymerization inhibitors (e.g., Paclitaxel, Docetaxel), DNA intercalators (e.g., Oxaliplatin), Topoisomerase II inhibitors (e.g., Irinotecan), and nucleotide analogs (e.g., Gemcitabine). These compounds work through quite distinct pathways, including those that bypass nuclear toxicity completely (taxols). Further, the CPT agents modulate chromatin across a wide variety of cancer cell line models with very distinct genetic/epigenetic profiles. Taken together, these findings demonstrate a mechanism of action rooted in limiting the ability of cells to acclimate to cytotoxic stress at non-replicative timescales.

### CPT Agents in the Context of Other Chromatin-Regulators

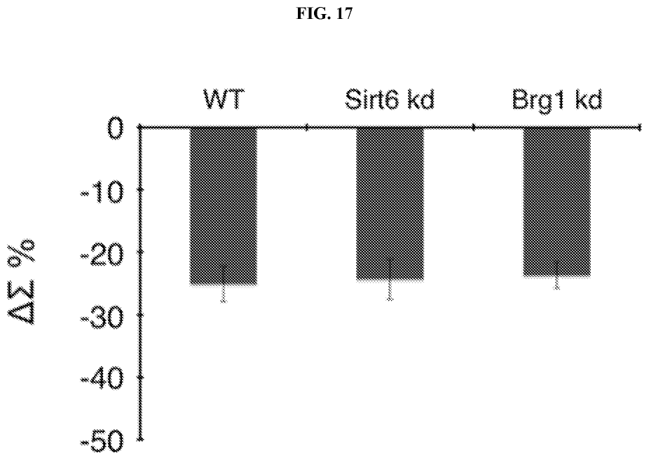

Most therapeutics development to date has been focused on targeting specific molecular pathways. Embodiments herein introduce normalization of the heterogeneity of physical supra-nucleosomal chromatin folding as a pathway. Global chromatin folding depends on physico-chemical intranuclear environment such molecular crowding. But some aspects of the global chromatin folding might also be regulated by a multitude of molecular pathways. Histone modifications, SWI/SNF, chromatin loop modifications (e.g. CTCF) are all known to affect chromatin structure at least to some extent. Data in multiple cell lines shows that targeting specific molecular pathways is less efficient at regulating chromatin folding compared to global physico-chemical effects (FIG. 16). The reason, most likely, is the very specificity of the pathway-specific effects, whereas physico-chemical mechanisms affect chromatin globally. And conversely, the identified CPT agents do not appear to act through pathway-specific chromatin remodeling (FIG. 17). The data herein demonstrate and support a new class of anti-cancer agents that work through physico-chemical mechanisms to achieve strong regulation of global chromatin folding.

### Example 4

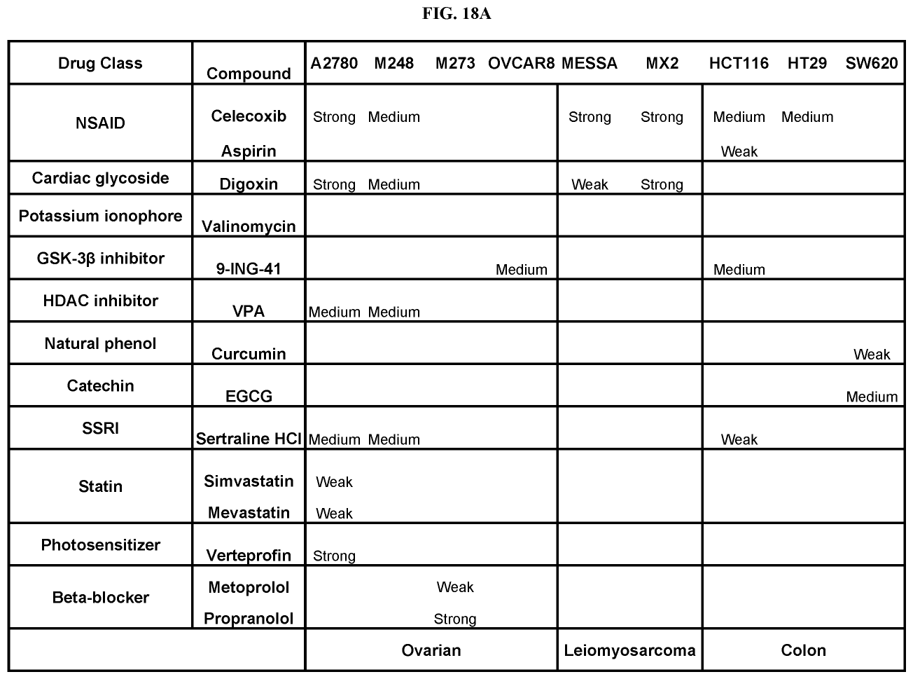

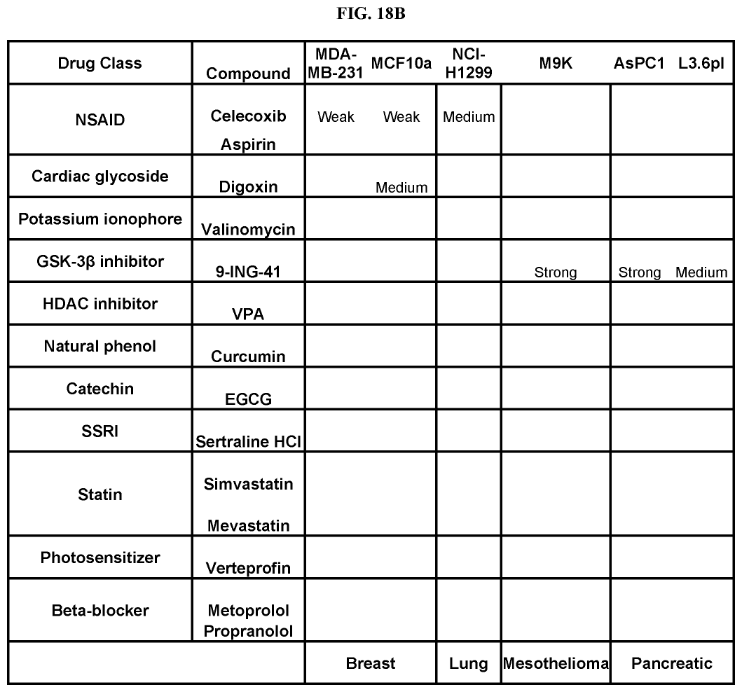

Experiments conducted during development of embodiments herein have demonstrated that administration of structurally and functionally diverse CPTs to a wide variety of cancer cell lines results in a reduction in chromatin folding/compaction/heterogeneity. A sample of these experiments is summarized in the tables of FIGS. 18A-B.

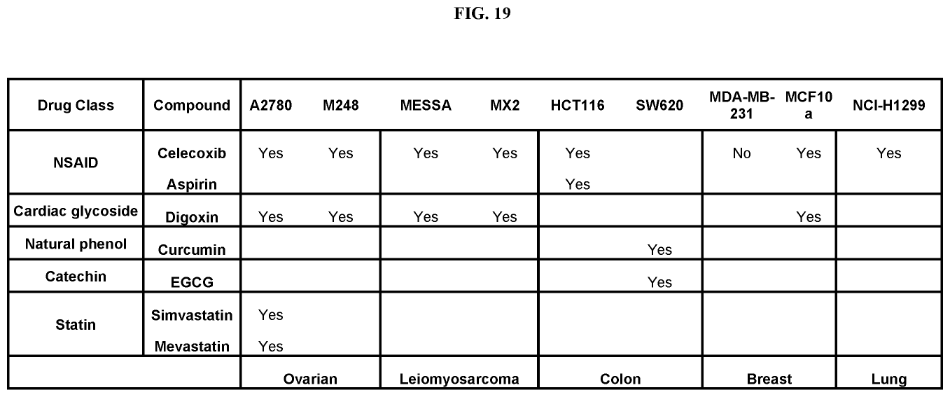

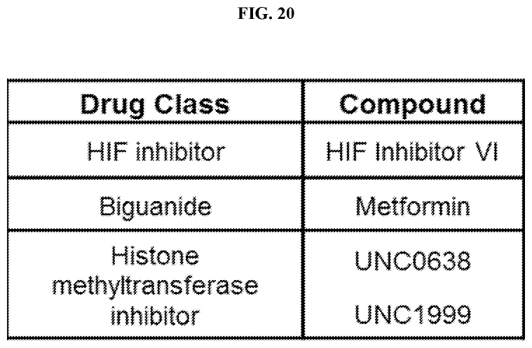

Experiments conducted during development of embodiments herein have demonstrated that administration of structurally and functionally diverse CPTs to a wide variety of cancer cell enhances cell death and/or allows for reduced dose of chemotherapeutic. A sample of these experiments is summarized in the table of FIG. 19.

Experiments conducted during development of embodiments herein have demonstrated that reduction in chromatin folding/compaction/heterogeneity strongly (FIGS. 18A-B) correlates with enhanced cell death and/or effective treatment at reduced chemotherapeutic dose (FIG. 19). Strong or medium reduction of chromatin heterogeneity by a CPT correlates with enhanced cell death and/or effective treatment at reduced chemotherapeutic dose in 100% of the compounds tested and in 100% of the cell lines. These experiments demonstrate that compounds that are capable of reducing chromatin folding/compaction/heterogeneity, regardless of their structure or other bioactivities, are useful in the treatment of cancer (e.g., for co-administration with chemotherapeutics or other agents).

All publications and patents mentioned above and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are cited above by number, are herein incorporated by reference in their entireties.
1a. Magee, J. A., Piskounova, E. & Morrison, S. J. Cancer stem cells: impact, heterogeneity, and uncertainty. Cancer Cell 21, 283-296, doi:10.1016/j.ccr.2012.03.003 (2012).
2a. Gerlinger, M. et al. Intratumor heterogeneity and branched evolution revealed by multiregion sequencing. N Engl J Med 366, 883-892, doi:10.1056/NEJMoa1113205 (2012).
3a. Swanton, C. Intratumor heterogeneity: evolution through space and time. Cancer Res 72, 4875-4882, doi:10.1158/0008-5472.CAN-12-2217 (2012).
4a. Burrell, R. A., McGranahan, N., Bartek, J. & Swanton, C. The causes and consequences of genetic heterogeneity in cancer evolution. Nature 501, 338-345, doi:10.1038/nature12625 (2013).
5a. Ling, S. et al. Extremely high genetic diversity in a single tumor points to prevalence of non-Darwinian cell evolution. Proc Natl Acad Sci USA 112, E6496-6505, doi:10.1073/pnas.1519556112 (2015).
6a. Easwaran, H., Tsai, H. C. & Baylin, S. B. Cancer epigenetics: tumor heterogeneity, plasticity of stem-like states, and drug resistance. Mol Cell 54, 716-727, doi:10.1016/j.molce1.2014.05.015 (2014).
7a. Tomasetti, C. & Vogelstein, B. Cancer etiology. Variation in cancer risk among tissues can be explained by the number of stem cell divisions. Science 347, 78-81, doi:10.1126/science.1260825 (2015).
8a. Sun, J. et al. Clonal dynamics of native haematopoiesis. Nature 514, 322-327, doi:10.1038/nature13824 (2014).
9a. Vogelstein, B. et al. Cancer genome landscapes. Science 339, 1546-1558, doi:10.1126/science.1235122 (2013).
10a. Levy, S. F., Ziv, N. & Siegal, M. L. Bet hedging in yeast by heterogeneous, age-correlated expression of a stress protectant. PLoS Biol 10, e1001325, doi:10.1371/journal.pbio.1001325 (2012).
11a. Breker, M., Gymrek, M. & Schuldiner, M. A novel single-cell screening platform reveals proteome plasticity during yeast stress responses. J Cell Biol 200, 839-850, doi:10.1083/jcb.201301120 (2013).
12a. Yadav, N. & Chandra, D. Mitochondrial and postmitochondrial survival signaling in cancer. Mitochondrion 16, 18-25, doi:10.1016/j.mito.2013.11.005 (2014).
13a. Lane, R. K., Hilsabeck, T. & Rea, S. L. The role of mitochondrial dysfunction in age-related diseases. Biochim Biophys Acta 1847, 1387-1400, doi:10.1016/j.bbabio.2015.05.021 (2015).
14a. Hay, N. Interplay between FOXO, TOR, and Akt. Biochim Biophys Acta 1813, 1965-1970, doi:10.1016/j.bbamcr.2011.03.013 (2011).
15a. Antico Arciuch, V. G., Elguero, M. E., Poderoso, J. J. & Carreras, M. C. Mitochondrial regulation of cell cycle and proliferation. Antioxid Redox Signal 16, 1150-1180, doi:10.1089/ars.2011.4085 (2012).
16a. Czabotar, P. E., Lessene, G., Strasser, A. & Adams, J. M. Control of apoptosis by the BCL-2 protein family: implications for physiology and therapy. Nat Rev Mol Cell Biol 15, 49-63, doi:10.1038/nrm3722 (2014).
17a. Welch, D. R. Tumor Heterogeneity-A ‘Contemporary Concept’ Founded on Historical Insights and Predictions. Cancer Res 76, 4-6, doi:10.1158/0008-5472.CAN-15-3024 (2016).
18a. Hanahan, D. & Weinberg, R. A. Hallmarks of cancer: the next generation. Cell 144, 646-674, doi:10.1016/j.cell.2011.02.013 (2011).
19a. Morgan, M. A. & Shilatifard, A. Chromatin signatures of cancer. Genes Dev 29, 238-249, doi:10.1101/gad.255182.114 (2015).
20a. Cancer Genome Atlas, N. Comprehensive molecular characterization of human colon and rectal cancer. Nature 487, 330-337, doi:10.1038/nature11252 (2012).
21a. Feinberg, A. P., Ohlsson, R. & Henikoff, S. The epigenetic progenitor origin of human cancer. Nat Rev Genet 7, 21-33, doi:10.1038/nrg1748 (2006).
22a. Bedin, V., Adam, R. L., de Sa, B. C., Landman, G. & Metze, K. Fractal dimension of chromatin is an independent prognostic factor for survival in melanoma. BMC Cancer 10, 260, doi:10.1186/1471-2407-10-260 (2010).
23a. Rasnick, D. Aneuploidy theory explains tumor formation, the absence of immune surveillance, and the failure of chemotherapy. Cancer Genet Cytogenet 136, 66-72 (2002).
24a. Brown, K. & Rufini, A. New concepts and challenges in the clinical translation of cancer preventive therapies: the role of pharmacodynamic biomarkers. Ecancermedicalscience 9, 601, doi:10.3332/ecancer.2015.601 (2015).
25a. Cherkezyan, L. et al. Nanoscale changes in chromatin organization represent the initial steps of tumorigenesis: a transmission electron microscopy study. BMC Cancer 14, 189, doi:10.1186/1471-2407-14-189 (2014).
26a. Stypula-Cyrus, Y. et al. HDAC up-regulation in early colon field carcinogenesis is involved in cell tumorigenicity through regulation of chromatin structure. PLoS One 8, e64600, doi:10.1371/journal.pone.0064600 (2013).
27a. Subramanian, H. et al. Nanoscale cellular changes in field carcinogenesis detected by partial wave spectroscopy. Cancer Res 69, 5357-5363, doi:10.1158/0008-5472.can-08-3895 (2009).
28a. Subramanian, H. et al. Optical methodology for detecting histologically unapparent nanoscale consequences of genetic alterations in biological cells. Proc Natl Acad Sci USA 105, 20118-20123, doi:10.1073/pnas.0804723105 (2008).
29a. Matsuda, H., Putzel, G. G., Backman, V. & Szleifer, I. Macromolecular crowding as a regulator of gene transcription. Biophys J 106, 1801-1810, doi:10.1016/j.bpj.2014.02.019 (2014).
30a. Tan, C., Saurabh, S., Bruchez, M. P., Schwartz, R. & Leduc, P. Molecular crowding shapes gene expression in synthetic cellular nanosystems. Nat Nanotechnol 8, 602-608, doi:10.1038/nnano.2013.132 (2013).
1b. Klar T A, Jakobs S, Dyba M, Egner A, & Hell S W (2000) Fluorescence microscopy with diffraction resolution barrier broken by stimulated emission. P Natl Acad Sci USA 97(15):8206-8210.
2b. Rust M J, Bates M, & Zhuang X W (2006) Sub-diffraction-limit imaging by stochastic optical reconstruction microscopy (STORM). Nature Methods 3(10):793-795.
3b. Betzig E, et al. (2006) Imaging intracellular fluorescent proteins at nanometer resolution. Science 313(5793):1642-1645.
4b. Jing C R & Cornish V W (2011) Chemical Tags for Labeling Proteins Inside Living Cells. Accounts Chem Res 44(9):784-792.
5b. Lelek M, et al. (2012) Superresolution imaging of HIV in infected cells with FlAsH-PALM.
P Natl Acad Sci USA 109(22):8564-8569.
6b. Mirny L A (2011) The fractal globule as a model of chromatin architecture in the cell. Chromosome Res 19(1):37-51.
7b. Cremer T, et al. (2000) Chromosome territories, interchromatin domain compartment, and nuclear matrix: An integrated view of the functional nuclear architecture. Crit Rev Eukar Gene 10(2):179-212.
8b. Eltsov M, Maclellan K M, Maeshima K, Frangakis A S, & Dubochet J (2008) Analysis of cryo-electron microscopy images does not support the existence of 30-nm chromatin fibers in mitotic chromosomes in situ. Proc Natl Acad Sci USA 105(50):19732-19737.
9b. Joti Y, et al. (2012) Chromosomes without a 30-nm chromatin fiber. Nucleus 3(5):404-410.
10b. Maeshima K, Imai R, Tamura S, & Nozaki T (2014) Chromatin as dynamic 10-nm fibers. Chromosoma 123(3):225-237.
11b. Boettiger A N, et al. (2016) Super-resolution imaging reveals distinct chromatin folding for different epigenetic states. Nature 529(7586):418-422.
12b. Lieberman-Aiden E, et al. (2009) Comprehensive mapping of long-range interactions reveals folding principles of the human genome. Science 326(5950):289-293.
13b. Dekker J, Marti-Renom M A, & Mirny L A (2013) Exploring the three-dimensional organization of genomes: interpreting chromatin interaction data. Nat Rev Genet 14(6):390-403.
14b. Botta M, Haider S, Leung I X Y, Lio P, & Mozziconacci J (2010) Intra- and inter-chromosomal interactions correlate with CTCF binding genome wide. Mol Syst Biot 6.
15b. Li L, et al. (2015) Widespread rearrangement of 3D chromatin organization underlies polycomb-mediated stress-induced silencing. Mol Cell 58(2):216-231.
16b. Hnisz D, et al. (2016) Activation of proto-oncogenes by disruption of chromosome neighborhoods. Science 351(6280):1454-1458.
17b. Cherkezyan L, et al. (2013) Interferometric Spectroscopy of Scattered Light Can Quantify the Statistics of Subdiffractional Refractive-Index Fluctuations. Physical Review Letters 111(3).
18b. Cherkezyan L, Subramanian H, & Backman V (2014) What structural length scales can be detected by the spectral variance of a microscope image? Opt Lett 39(15):4290-4293.
19b. J. E. Chandler Y S-C, L Almassalha, G Bauer, L Bowen, H Subramanian, I Szleifer, and V Backman (2016) Colocalization of cellular nanostructure using confocal fluorescence and partial wave spectroscopy. Journal of Biophotonics.
20b. Cherkezyan L, et al. (2014) Nanoscale changes in chromatin organization represent the initial steps of tumorigenesis: a transmission electron microscopy study. BMC Cancer 14:189.
21b. Stypula-Cyrus Y, et al. (2013) HDAC up-regulation in early colon field carcinogenesis is involved in cell tumorigenicity through regulation of chromatin structure. PLoS One 8(5):e64600.
22b. Kim J S, Pradhan P, Backman V, & Szleifer I (2011) The influence of chromosome density variations on the increase in nuclear disorder strength in carcinogenesis. Phys Biol 8(1):015004.
23b. Chandler J E, Cherkezyan L, Subramanian H, & Backman V (2016) Nanoscale refractive index fluctuations detected via sparse spectral microscopy. Biomed. Opt. Express 7(3):883-893.
24b. Rogakou E P, Nieves-Niera W, Boon C, Pommier Y, & Bonner W M (1998) Histone H2AX serine-139 phosphorylation is induced by the introduction of the initial breaks into DNA as a result of the apoptotic endonuclease. Mol Biol Cell 9:109A-109A.
25b. Burma S, Chen B P, Murphy M, Kurimasa A, & Chen D J (2001) ATM phosphorylates histone H2AX in response to DNA double-strand breaks. J Biol Chem 276(45):42462-42467.
26b. Olive P L (2004) Detection of DNA damage in individual cells by analysis of histone H2AX phosphorylation. Method Cell Biol 75:355-373.
27b. Banath J P & Olive P L (2003) Expression of phosphorylated histone H2A X as a surrogate of cell killing by drugs that create DNA double-strand breaks. Cancer Res 63(15):4347-4350.
28b. Cohen S M & Lippard S J (2001) Cisplatin: From DNA damage to cancer chemotherapy. Prog Nucleic Acid Re 67:93-130.
29b. Burgess R C, Burman B, Kruhlak M J, & Misteli T (2014) Activation of DNA damage response signaling by condensed chromatin. Cell Rep 9(5):1703-1717.
30b. Johnson S A, You Z, & Hunter T (2007) Monitoring ATM kinase activity in living cells. DNA Repair (Amst) 6(9):1277-1284.
31b. Beerman T A, et al. (1992) Effects of Analogs of the DNA Minor Groove Binder Hoechst-33258 on Topoisomerase-Ii and Topoisomerase-I Mediated Activities. Biochim Biophys Acta 1131(1):53-61.
32b. Zhang X B, Chen J, Davis B, & Kiechle F (1999) Hoechst 33342 induces apoptosis in HL-60 cells and inhibits topoisomerase I in vivo. Arch Pathol Lab Med 123(10):921-927.
33b. Kruhlak M J, et al. (2006) Changes in chromatin structure and mobility in living cells at sites of DNA double-strand breaks. J Cell Biol 172(6):823-834.
34b. Daugas E, et al. (2000) Mitochondrio-nuclear translocation of AIF in apoptosis and necrosis. Faseb J 14(5):729-739.
35b. Lakhani S A, et al. (2006) Caspases 3 and 7: Key mediators of mitochondrial events of apoptosis. Science 311(5762):847-851.
36b. Martinez-Pastor B, Cosentino C, & Mostoslaysky R (2013) A Tale of Metabolites: The Cross-Talk between Chromatin and Energy Metabolism. Cancer Discov 3(5):497-501.
37b. Lum J J, et al. (2007) The transcription factor HIF-1 alpha plays a critical role in the growth factor-dependent regulation of both aerobic and anaerobic glycolysis. Gene Dev 21(9):1037-1049.
38b. Liu X S, Little J B, & Yuan Z M (2015) Glycolytic metabolism influences global chromatin structure. Oncotarget 6(6):4214-4225.
39b. Visvanathan A, et al. (2013) Modulation of Higher Order Chromatin Conformation in Mammalian Cell Nuclei Can Be Mediated by Polyamines and Divalent Cations. Plos One 8(6).
40b. Tice R R, Hook G G, Donner M, McRee D I, & Guy A W (2002) Genotoxicity of radiofrequency signals. I. Investigation of DNA damage and micronuclei induction in cultured human blood cells. Bioelectromagnetics 23(2):113-126.
41b. Bancaud A, et al. (2009) Molecular crowding affects diffusion and binding of nuclear proteins in heterochromatin and reveals the fractal organization of chromatin. Embo J 28(24):3785-3798.
42b. Papantonis A & Cook P R (2010) Genome architecture and the role of transcription. Curr Opin Cell Biol 22(3):271-276.
43b. Papantonis A, et al. (2010) Active RNA Polymerases: Mobile or Immobile Molecular Machines? Plos Biol 8(7).
44b. Williamson I, et al. (2014) Spatial genome organization: contrasting views from chromosome conformation capture and fluorescence in situ hybridization. Gene Dev 28(24):2778-2791.
45b. Fudenberg G, Getz G, Meyerson-M, & Mirny L A (2011) High order chromatin architecture shapes the landscape of chromosomal alterations in cancer. Nat Biotechnol 29(12):1109-U1175.
46b. Katada S, Imhof A, & Sassone-Corsi P (2012) Connecting Threads: Epigenetics and Metabolism. Cell 148(1-2):24-28.
47b. Azuara V, et al. (2006) Chromatin signatures of pluripotent cell lines. Nat Cell Biol 8(5):532-U189.
48b. Hajkova P, et al. (2008) Chromatin dynamics during epigenetic reprogramming in the mouse germ line. Nature 452(7189):877-U876.
1c. H. Matsuda, G. G. Putzel, V. Backman, and I. Szleifer, “Macromolecular Crowding as a Regulator of Gene Transcription,” Biophysical Journal 106, 1801-1810 (2014).
2c. S. T. Pan, Z. L. Li, Z. X. He, J. X. Qiu, and S. F. Zhou, “Molecular mechanisms for tumour resistance to chemotherapy,” Clinical and Experimental Pharmacology and Physiology 43, 723-737 (2016).
3c. A. N. Boettiger, B. Bintu, J. R. Moffitt, S. Y. Wang, B. J. Beliveau, G. Fudenberg, M. Imakaev, L. A. Mirny, C. T. Wu, and X. W. Zhuang, “Super-resolution imaging reveals distinct chromatin folding for different epigenetic states,” Nature 529, 418-+(2016).
4c. L. A. Garraway, and P. A. Janne, “Circumventing Cancer Drug Resistance in the Era of Personalized Medicine,” Cancer Discovery 2, 214-226 (2012).
5c. P. L. Bedard, A. R. Hansen, M. J. Ratain, and L. L. Siu, “Tumour heterogeneity in the clinic,”
Nature 501, 355-364 (2013).
6c. R. Fisher, L. Pusztai, and C. Swanton, “Cancer heterogeneity: implications for targeted therapeutics,” British Journal of Cancer 108, 479-485 (2013).
7c. S. M. Shaffer, M. C. Dunagin, S. R. Torborg, E. A. Torre, B. Emert, C. Krepler, M. Beqiri, K. Sproesser, P. A. Brafford, M. Xiao, E. Eggan, I. N. Anastopoulos, C. A. Vargas-Garcia, A. Singh, K. L. Nathanson, M. Herlyn, and A. Raj, “Rare cell variability and drug-induced reprogramming as a mode of cancer drug resistance,” Nature advance online publication (2017).
8c. M. Kaern, T. C. Elston, W. J. Blake, and J. J. Collins, “Stochasticity in gene expression: From theories to phenotypes,” Nature Reviews Genetics 6, 451-464 (2005).
9c. R. A. Gatenby, J. J. Cunningham, and J. S. Brown, “Evolutionary triage governs fitness in driver and passenger mutations and suggests targeting never mutations,” Nature Communications 5 (2014).
10c. L. M. Almassalha, G. M. Bauer, J. E. Chandler, S. Gladstein, I. Szleifer, H. K. Roy, and V. Backman, “The Greater Genomic Landscape: The Heterogeneous Evolution of Cancer,” Cancer Research 76, 5605-5609 (2016).
11c. L. M. Almassalha, G. M. Bauer, W. Wu, L. Cherkezyan, D. Zhang, A. Kendra, S. Gladstein, J. E. Chandler, D. VanDerway, N. Seagle, A. Ugolkov, D. D. Billadeau, T. V. O'Halloran, A. P. Mazar, H. K. Roy, I. Szleifer, S. Shahabi, and V. Backman, “Macrogenomic engineering by manipulating variations in chromatin folding,” Nature Biomedical Engineering, under review.
12c. L. M. Almassalha, A. Tiwari, P. T. Ruhoff, Y. Stypula-Cyrus, L. Cherkezyan, H. Matsuda, M. A. Dela Cruz, J. E. Chandler, C. White, C. Maneval, H. Subramanian, I. Szleifer, H. K. Roy, and V. Backman, “The Global Relationship between Chromatin Physical Topology, Fractal Structure, and Gene Expression,” Scientific Reports 7, 41061 (2017).
13c. E. Lieberman-Aiden, N. L. van Berkum, L. Williams, M. Imakaev, T. Ragoczy, A. Telling, I. Amit, B. R. Lajoie, P. J. Sabo, M. O. Dorschner, R. Sandstrom, B. Bernstein, M. A. Bender, M. Groudine, A. Gnirke, J. Stamatoyannopoulos, L. A. Mirny, E. S. Lander, and J. Dekker, “Comprehensive mapping of long-range interactions reveals folding principles of the human genome,” Science 326, 289-293 (2009).
14c. S. S. Rao, M. H. Huntley, N. C. Durand, E. K. Stamenova, I. D. Bochkov, J. T. Robinson, A. L. Sanborn, I. Machol, A. D. Omer, E. S. Lander, and E. L. Aiden, “A 3D map of the human genome at kilobase resolution reveals principles of chromatin looping,” Cell 159, 1665-1680 (2014).
15c. L. M. Almassalha, G. M. Bauer, J. E. Chandler, S. Gladstein, L. Cherkezyan, Y. Stypula-Cyrus, S. Weinberg, D. Zhang, P. Thusgaard Ruhoff, H. K. Roy, H. Subramanian, N. Chandel, I. Szleifer, and V. Backman, “Label-free imaging of the native, living cellular nanoarchitecture using partial-wave spectroscopic microscopy,” PNAS (2016).
16c. B. Dong, L. M. Almassalha, Y. Stypula-Cyrus, B. E. Urban, J. E. Chandler, T.-Q. Nguyen, C. Sun, H. F. Zhang, and V. Backman, “Superresolution intrinsic fluorescence imaging of chromatin utilizing native, unmodified nucleic acids for contrast,” Proceedings of the National Academy of Sciences 113, 9716-9721 (2016).
17c. R. L. Siegel, K. D. Miller, S. A. Fedewa, D. J. Ahnen, R. G. Meester, A. Barzi, and A. Jemal, “Colorectal cancer statistics, 2017,” CA Cancer J Clin., 1-17 (2017).
18c. J. Bachl, C. Carlson, V. Gray-Schopfer, M. Dessing, and C. Olsson, “Increased transcription levels induce higher mutation rates in a hypermutating cell line,” Journal of Immunology 166, 5051-5057 (2001).
19c. D. A. Yushchenko, and M. P. Bruchez, “Tailoring Fluorescent Labels for Far-Field Nanoscopy,” Springer (2012).
20c. L. Cherkezyan, I. Capoglu, H. Subramanian, J. D. Rogers, D. Damania, A. Taflove, and V. Backman, “Interferometric Spectroscopy of Scattered Light Can Quantify the Statistics of Subdiffractional Refractive-Index Fluctuations,” Physical Review Letters 111 (2013).
21c. L. M. Almassalha, G. M. Bauer, J. E. Chandler, S. Gladstein, L. Cherkezyan, Y. Stypula-Cyrus, S. Weinberg, D. Zhang, P. T. Ruhoff, H. K. Roy, H. Subramanian, N. S. Chandel, I. Szleifer, and V. Backman, “Label-free imaging of the native, living cellular nanoarchitecture using partial-wave spectroscopic microscopy,” Proceedings of the National Academy of Sciences of the United States of America 113, E6372-E6381 (2016).
22c. E. Lieberman-Aiden, N. L. Van Berkum, L. Williams, M. Imakaev, T. Ragoczy, A. Telling, I. Amit, B. R. Lajoie, P. J. Sabo, and M. O. Dorschner, “Comprehensive mapping of long-range interactions reveals folding principles of the human genome,” science 326, 289-293 (2009).
23c. S. S. Rao, M. H. Huntley, N. C. Durand, E. K. Stamenova, I. D. Bochkov, J. T. Robinson, A. L. Sanborn, I. Machol, A. D. Omer, and E. S. Lander, “A 3D map of the human genome at kilobase resolution reveals principles of chromatin looping,” Cell 159, 1665-1680 (2014).
24c. A. N. Boettiger, B. Bintu, J. R. Moffitt, S. Wang, B. J. Beliveau, G. Fudenberg, M. Imakaev, L. A. Mirny, C.-t. Wu, and X. Zhuang, “Super-resolution imaging reveals distinct chromatin folding for different epigenetic states,” Nature 529, 418-422 (2016).
25c. B. Dong, L. M. Almassalha, Y. Stypula-Cyrus, B. E. Urban, J. E. Chandler, C. Sun, H. F. Zhang, and V. Backman, “Superresolution intrinsic fluorescence imaging of chromatin utilizing native, unmodified nucleic acids for contrast,” Proceedings of the National Academy of Sciences 113, 9716-9721 (2016).
26c. J. Dostie, T. A. Richmond, R. A. Arnaout, R. R. Selzer, W. L. Lee, T. A. Honan, E. D. Rubio, A. Krumm, J. Lamb, and C. Nusbaum, “Chromosome Conformation Capture Carbon Copy (5C): a massively parallel solution for mapping interactions between genomic elements,” Genome research 16, 1299-1309 (2006).
27c. W. Wu, A. J. Radosevich, A. Eshein, J. Yi, L. Cherkezyan, H. K. Roy, I. Szleifer, and V. Backman, “Using electron microscopy to calculate optical properties of biological samples,” Biomedical Optics Express 7, 4749-4762 (2016).
28c. H. Subramanian, H. K. Roy, P. Pradhan, M. J. Goldberg, J. Muldoon, R. E. Brand, C. Sturgis, T. Hensing, D. Ray, and A. Bogojevic, “Nanoscale cellular changes in field carcinogenesis detected by partial wave spectroscopy,” Cancer research 69, 5357-5363 (2009).
29c. V. Levi, Q. Ruan, and E. Gratton, “3-D particle tracking in a two-photon microscope: application to the study of molecular dynamics in cells,” Biophysical journal 88, 2919-2928 (2005).
30c. A. Bancaud, C. Lavelle, S. Huet, and J. Ellenberg, “A fractal model for nuclear organization: current evidence and biological implications,” Nucleic acids research, gks586 (2012).
31c. A. Bancaud, S. Huet, N. Daigle, J. Mozziconacci, J. Beaudouin, and J. Ellenberg, “Molecular crowding affects diffusion and binding of nuclear proteins in heterochromatin and reveals the fractal organization of chromatin,” The EMBO journal 28, 3785-3798 (2009).
32c. L. A. Mirny, “The fractal globule as a model of chromatin architecture in the cell,” Chromosome Research 19, 37-51 (2011).
33c. B. B. Mandelbrot, The fractal geometry of nature (Macmillan, 1983).
34c. G. Fudenberg, M. Imakaev, C. Lu, A. Goloborodko, N. Abdennur, and L. A. Mirny, “Formation of chromosomal domains by loop extrusion,” Cell reports 15, 2038-2049 (2016).
35c. R. Sachs, G. Van Den Engh, B. Trask, H. Yokota, and J. Hearst, “A random-walk/giant-loop model for interphase chromosomes,” Proceedings of the National Academy of Sciences 92, 2710-2714 (1995).
36c. J. S. Kim, V. Backman, and I. Szleifer, “Crowding-induced structural alterations of random-loop chromosome model,” Physical review letters 106, 168102 (2011).
37c. M. Bohn, and D. W. Heermann, “Diffusion-driven looping provides a consistent framework for chromatin organization,” PloS one 5, e12218 (2010).
38c. A. Bancaud, S. Huet, N. Daigle, J. Mozziconacci, J. Beaudouin, and J. Ellenberg, “Molecular crowding affects diffusion and binding of nuclear proteins in heterochromatin and reveals the fractal organization of chromatin,” Embo Journal 28, 3785-3798 (2009).
39c. H. T. Lynch, M. Rendell, T. G. Shaw, P. Silberstein, and B. T. Ngo, “Commentary on Almassalha et al., “The Greater Genomic Landscape: The Heterogeneous Evolution of Cancer”,” Cancer Research 76 (2016).
40c. Y. Stypula-Cyrus, D. Damania, D. P. Kunte, M. Dela Cruz, H. Subramanian, H. K. Roy, and V. Backman, “HDAC Up-Regulation in Early Colon Field Carcinogenesis Is Involved in Cell Tumorigenicity through Regulation of Chromatin Structure,” Plos One 8 (2013).

1. A composition comprising a chromatin protective therapeutic (CPT), wherein the CPT reduces chromatin heterogeneity and/or inhibits increases in chromatin heterogeneity.

2. The composition of claim 1, wherein the CPT is formulated as a pharmaceutical composition.

3. The composition of claim 1, further comprising a cancer chemotherapeutic.

4. A method of treating and/or preventing cancer and/or tumor formation in a subject comprising administering to the subject a chromatin protective therapeutic (CPT), wherein the CPT reduces chromatin heterogeneity and/or transcriptional heterogeneity, and/or inhibits increases in chromatin heterogeneity and/or transcriptional heterogeneity, thereby reducing the likelihood of cancer and/or tumor formation in the subject.

5. The method of claim 4, wherein the CPT is co-administered with a second agent.

6. The method of claim 5, wherein the second agent is a chemotherapeutic agent or immunotherapeutic agent.

7. The method of claim 5, wherein the second agent is administered at a dose that is less that the therapeutic dose for the second agent alone.

8. The method of claim 5, wherein the CPT and the second agent are administered simultaneously.

9. The method of claim 8, wherein the CPT and the second agent are co-formulated.

10. The method of claim 5, wherein the CPT and the second agent are administered separately.

11. The method of claim 10, wherein the CPT is administered at least 24 hours prior to the second agent.

12. The method of claim 10, wherein the second agent is administered at least 24 hours prior to the CPT.

13. A method of treating and/or preventing chemotherapeutic resistance and/or immune evasion in a subject being treated for cancer comprising administering to the subject a chromatin protective therapeutic (CPT), wherein the CPT reduces chromatin heterogeneity and/or transcriptional heterogeneity, and/or inhibits increases in chromatin heterogeneity and/or transcriptional heterogeneity, thereby reducing the likelihood of cancer and/or tumor formation in the subject.

14. The method of claim 13, wherein the CPT is co-administered with a second agent.

15. The method of claim 13, wherein the second agent is administered at a dose that is less that the therapeutic dose for the second agent alone.

16. The method of claim 14, wherein the second agent is a chemotherapeutic agent or immunotherapeutic agent.

17. The method of claim 14, wherein the CPT and the second agent are administered simultaneously.

18. The method of claim 17, wherein the CPT and the second agent are co-formulated.

19. The method of claim 14, wherein the CPT and the second agent are administered separately.

20. The method of claim 19, wherein the CPT is administered at least 24 hours prior to the second agent.

21. The method of claim 19, wherein the second agent is administered at least 24 hours prior to the CPT.

22. A method of monitoring the treatment and/or prevention of cancer, the likelihood of a subject developing cancer, and/or the progression/regression of cancer in a subject, comprising:
(a) measuring chromatin heterogeneity in a population of cells from the subject at a first time point;
(b) measuring the chromatin heterogeneity in a similar population of cells from the subject at a second time point;
(c) comparing the chromatin heterogeneity at the first and second time points, wherein:
(i) a decrease in chromatin heterogeneity indicates cancer is being treated or prevented successfully, the likelihood of the subject developing cancer is reduced, and/or cancer is not progressing/is regressing in the subject; or
(ii) an increase in chromatin heterogeneity indicates cancer not being treated or prevented successfully, the likelihood of the subject developing cancer is increased, and/or cancer is progressing/is not regressing in the subject.

23. The method of claim 22, further comprising administering a chromatin protective therapeutic between steps (a) and (b).

24. The method of claim 22, wherein the population of cells is obtained by biopsy.

25. The method of claim 22, wherein chromatic heterogeneity is measured by live-cell partial wave spectroscopic (PWS) microscopy.

26. The method of claim 22, wherein chromatin heterogeneity correlates with transcriptional heterogeneity.

27. A method of identifying a chromatin protective therapeutic (CPT), comprising:
(a) measuring chromatin heterogeneity in a population of cells at a first time point;
(b) administering a test agent to the population of cells;
(c) measuring the chromatin heterogeneity in the population of cells at a second time point;
(d) comparing the chromatin heterogeneity at the first and second time points, wherein a decrease in chromatin heterogeneity indicates the test agent is active as a CPT.

28. The method of claim 27, wherein chromatic heterogeneity is measured by live-cell partial wave spectroscopic microscopy.

29. A method of screening a library of test agents to identify a chromatin protective therapeutic (CPT), comprising:
(a) measuring chromatin heterogeneity of multiple populations of cells at a first time point;
(b) administering different test agents to each of the populations of cells;
(c) measuring the chromatin heterogeneity in each of the population of cells at a second time point;
(d) comparing the chromatin heterogeneity at the first and second time points, wherein a decrease in chromatin heterogeneity indicates a test agent is active as a CPT.

30. The method of claim 29, wherein chromatic heterogeneity is measured by high-throughput live-cell partial wave spectroscopic microscopy.
